(19) United States
(12) Patent Application Publication
(10) Pub. No.: US 2023/0329537 A1
(43) Pub. Date: Oct. 19, 2023

(54) INSERTION APPARATUS, AND METHOD OF ILLUMINATING INSIDE OF SUBJECT

(71) Applicant: OLYMPUS MEDICAL SYSTEMS CORP., Tokyo (JP)

(72) Inventor: Satoshi HORIE, Tokyo (JP)

(73) Assignee: OLYMPUS MEDICAL SYSTEMS CORP., Tokyo (JP)

(21) Appl. No.: 18/133,072

(22) Filed: Apr. 11, 2023

Related U.S. Application Data
(60) Provisional application No. 63/332,080, filed on Apr. 18, 2022.

(51) Int. Cl. A61B 1/07; A61B 1/00; A61B 1/06; A61B 1/018

(52) U.S. Cl. CPC A61B 1/07; A61B 1/00117; A61B 1/0669; A61B 1/00096; A61B 1/0646; A61B 1/018

(57) ABSTRACT

An insertion apparatus includes: an insertion portion including, a first conduit having a first opening, an illumination chamber having a second opening, an optical fiber configured to guide and radiate an illumination light, and an illumination lens. The first conduit is in fluid communication with an outside of the insertion portion via the first opening. The illumination chamber is configured to fluidly communicate with a peripheral part of the first conduit via the second opening. A distal end of the optical fiber is located inside of the illumination chamber. The illumination lens is configured to radiate the illumination light to the outside of the insertion portion.

### RELATED APPLICATION DATA

This application is based on and claims priority under 37 U.S.C. § 119 to U.S. Provisional Application No. 63/332,080 filed on Apr. 18, 2022, the entire contents of which are incorporated herein by reference.

### BACKGROUND OF THE DISCLOSURE

The present disclosure relates to an insertion apparatus such as an endoscope and a method of illuminating an inside of a subject with the insertion apparatus.

Conventionally, insertion apparatuses such as endoscopes have been widely used, for example, in medical and industrial fields. A typical endoscope or the like includes an insertion portion in an elongated tubular shape and a distal end portion of the insertion portion (hereinafter simply referred to as a distal end portion) provided at a distal end of the insertion portion.

The distal end portion is provided with, for example, an image pickup unit and an illumination unit.

In the conventional endoscope or the like with such a configuration, for example, the insertion portion is inserted into a body cavity of a subject (living body), and image data of an object to be observed or treated (lesion, etc.) can be acquired with the image pickup unit while the object to be observed or treated (lesion, etc.) is irradiated with illumination light from the illumination unit.

Images based on the image data acquired in such a way can be displayed with a display monitor or the like.

With the endoscope thus used, a target object in the body cavity can be observed, examined, or treated, for example.

In addition, conventional insertion apparatuses such as endoscopes may be used under an underwater environment.

More specifically, for example, an endoscope to be used for observing or treating the inside of an organ in the urinary system has an insertion portion with an extremely small diameter.

In addition, such an endoscope includes a watertight structure in consideration of being able to be used under an underwater environment.

For the illumination units used in conventional general endoscopes, etc., for example, the illumination units each including an illumination lens and an illumination member (optical fiber cable, etc.) are generally put into practical use. For the illumination units for endoscope or the like of such type, units of various types have been proposed, for example, in Japanese Patent No. 6281028.

The illumination unit disclosed in Japanese Patent No. 6281028 is an illumination optical system for endoscope configured to be applied to endoscopes and the like used under an underwater environment.

The illumination unit is configured so that the distal end portion (light emitting portion) of the optical fiber cable is placed to be inserted into a non-through hole formed in the distal end portion (transparent resin member) of the endoscope.

Here, an air layer is provided in a gap formed between the distal end side inner surface of the non-through hole and the distal end surface of the optical fiber cable.

In the illumination unit thus configured, the curved shape of the distal end side inner surface of the non-through hole and the curved shape of the outer surface of the distal end portion are devised.

The configuration achieves obtaining illumination light distribution characteristics over a larger irradiation range when the illumination light emitted from the distal end surface of the optical fiber cable is radiated forward.

In the case, in the illumination unit described in the above patent publication, the air layer in the non-through hole and the inner and outer surfaces of the distal end portion function as an illumination lens.

In the configuration, in order to ensure and maintain the function of the illumination lens, the inside of the non-through hole must be sealed in a watertight manner in a state in which the distal end portion of the optical fiber cable is inserted.

In addition, there is demands for downsizing and reduction in diameters for insertion apparatuses such as endoscopes.

As endoscopes are downsized and reduced in diameters, for example, various component members to be used (for example, optical fiber cables in illumination units), which are disposed inside the distal end portions, are inevitably downsized and reduced in diameters.

In addition, the internal shapes of the distal end portion itself, in which the downsized and smaller-diameter component members are disposed, tend to become more complicated.

On the other hand, the insertion portions of conventional endoscopes or the like are each provided with a plurality of conduits inserted through the insertion portion.

The conduits include, for example, a liquid feeding conduit for perfusion to send liquids such as physiological saline from the outside into the body cavity.

Alternatively, the conduits include a suction conduit used to suction an object to be suctioned such as a liquid in the body cavity (a liquid sent into the body cavity, blood caused by bleeding, etc.), to discharge the object out of the body cavity.

Alternatively, the conduits include a treatment instrument insertion conduit for introducing a treatment instrument into a target site (site to be observed or treated such as a lesion) in the body cavity.

Note that some of the conduits in the insertion portion are configured to be used for some of various purposes.

### SUMMARY OF THE DISCLOSURE

An insertion apparatus according to one aspect of the present disclosure includes: an insertion portion including, a first conduit having a first opening, an illumination chamber having a second opening, an optical fiber configured to guide and radiate an illumination light, and an illumination lens. The first conduit is in fluid communication with an outside of the insertion portion via the first opening. The illumination chamber is configured to fluidly communicate with a peripheral part of the first conduit via the second opening. A distal end of the optical fiber is located inside of the illumination chamber. The illumination lens is configured to radiate the illumination light to the outside of the insertion portion.

An insertion apparatus according to one aspect of the present disclosure includes: an insertion portion configured for insertion into a subject. The insertion portion includes: a peripheral surface, an illumination chamber and an opening, an optical fiber configured to guide and radiate an illumination light, and an illumination window configured to radiate the illumination light to the outside of the insertion portion. The opening is located in the peripheral surface and wherein the illumination chamber is in fluid communication with an outside of the insertion portion via the opening. A distal end of the optical fiber is located inside the illumination chamber.

A medical kit according to one aspect of the present disclosure includes: an insertion apparatus including an insertion portion configured for insertion into a subject. The insertion portion includes: an illumination chamber, an optical fiber configured to guide and radiate an illumination light, and an illumination window configured to radiate the illumination light to an outside of the insertion portion. A distal end of the optical fiber is located inside of the illumination chamber. The illumination window is spaced apart from the distal end of the optical fiber to form a space.

### BRIEF DESCRIPTION OF THE DRAWINGS

FIG. 1 is a schematic configuration diagram showing an endoscope system including an endoscope that is an insertion apparatus according to individual embodiments of the present disclosure.

FIG. 2 is an enlarged perspective view of a main part showing an enlarged distal end portion of an insertion portion in an endoscope that is an insertion apparatus of a first embodiment of the present disclosure.

FIG. 3 is a plan view of a distal end surface of the distal end portion of FIG. 2 as seen in a direction of an arrow [3] in FIG. 2.

FIG. 4 is a cross-sectional view taken along a line [4]-[4] in FIG. 3.

FIG. 5 is an explanatory diagram of actions of the endoscope of the first embodiment of the present disclosure in illuminating an inside of a body cavity of a subject.

FIG. 6 is an enlarged perspective view of a main part showing an enlarged distal end portion of an insertion portion in an endoscope that is an insertion apparatus according to a second embodiment of the present disclosure.

FIG. 7 is a cross-sectional view of the distal end portion in FIG. 6 taken along a plane indicated by arrows [7] in FIG. 6.

FIG. 8 is a cross-sectional view showing an enlarged distal end portion of an insertion portion in an endoscope that is an insertion apparatus according to a third embodiment of the present disclosure.

FIG. 9 is an enlarged cross-sectional view of a main part showing an enlarged cut-out area indicated by an arrow mark [9] in FIG. 8.

FIG. 10 is a cross-sectional view showing a modification of the third embodiment of the present disclosure.

FIG. 11 is a plan view of a distal end surface of a distal end portion of an insertion portion in an endoscope that is an insertion apparatus of a fourth embodiment of the present disclosure.

FIG. 12 is a cross-sectional view taken along a line [12]-[12] in FIG. 11.

FIG. 13 is an explanatory diagram of actions in the endoscope of the fourth embodiment of the present disclosure in illuminating an inside of a body cavity of a subject.

FIG. 14 is a plan view of a distal end surface of a distal end portion of an insertion portion in an endoscope that is an insertion apparatus of a fifth embodiment of the present disclosure.

FIG. 15 is a cross-sectional view taken along a line [15]-[15] in FIG. 14.

FIG. 16 is a plan view of a side surface of the distal end portion as seen in a direction of an arrow mark [16] in FIG. 14.

FIG. 17 is a side view showing a modification of a flow path opening on the distal end portion of the fifth embodiment of the present disclosure.

FIG. 18 is a cross-sectional view showing an enlarged distal end portion of an insertion portion in an endoscope that is an insertion apparatus according to a sixth embodiment of the present disclosure.

FIG. 19 is a plan view showing an enlarged distal end surface of a distal end portion of an insertion portion in an endoscope that is an insertion apparatus according to a seventh embodiment of the present disclosure.

FIG. 20 is a cross-sectional view taken along a line [20]-[20] in FIG. 19.

FIG. 21 is an enlarged cross-sectional view of a main part showing an enlarged cut-out area indicated by an arrow mark [21] in FIG. 20.

### DETAILED DESCRIPTION

In general, as endoscopes are downsized and reduced in diameters, and the distal end portion itself of the insertion portion and various component members disposed at the distal end portion are downsized and reduced in diameters. Accordingly, the degree of difficulty in assembling the endoscopes tends to increase gradually.

For example, in assembly of an optical fiber cable or the like to a distal end portion of an insertion portion of an endoscope, the assembly obviously is more difficult as the optical fiber cable or the like has a smaller diameter.

In addition, after the optical fiber cable or the like is assembled to a position of the distal end portion, the distal end portion of the optical fiber cable or the like may be sealed from the outside in a watertight manner.

For the purpose, an adhesive or the like is applied, for example, but the application or injection of the adhesive or the like into a gap space or the like is more difficult as the gap is smaller.

As described above, when a conventional endoscope is downsized and reduced in the diameter, difficulty in assembly increases. The increased difficulty in assembly may involve increased difficulty in ensuring watertightness around a light emitting portion in an illumination unit, for example.

In particular, in a case in which conventional endoscopes, capable of perfusion or suction through conduits and to be used under an underwater environment, is downsized and reduced in the diameter, watertightness may not be ensured due to the difficulty in assembly. In such a case, for example, there may be flooding around a light emitting portion of an illumination unit.

If water floods around the light emitting portion of the illumination unit, the illumination light distribution characteristics become unstable.

If the illumination light distribution characteristics become unstable, there arises a problem such that it becomes difficult to effectively and efficiently irradiate an object to be observed or treated with good illumination light.

Embodiments of the present disclosure described below each makes it possible to provide an insertion apparatus such as an endoscope capable of perfusion or suction through a conduit and to be used under an underwater environment with a configuration that eliminates the need to consider the difficulty of ensuring watertightness around an illumination unit due to downsizing and reduction in the diameter.

In addition, it is possible to provide an insertion apparatus such as an endoscope that can be used under an underwater environment while maintaining stable and illumination light distribution characteristics.

Also, it is possible to provide a method of illuminating an inside of a subject with the insertion apparatus.

The present disclosure is to be described below with reference to illustrated embodiments.

Drawings to be used in the following descriptions each schematically shows components, and indicates each component in a size that can be recognized on the drawings. Therefore, there are cases in which the dimensional relationships, scales, etc., among individual members are shown differently for each component.

Accordingly, the present disclosure is not limited to only the illustrated forms shown in the individual drawings regarding the quantity of the individual components, the shape of individual components, the ratio of the size in the individual components, the relative positional relationship among the individual components and so on.

Note that, in each embodiment shown below, an example of the insertion apparatus of the present disclosure is illustrated with, for example, an endoscope used for a case in which the object to be observed or treated is the inside of an organ in the urinary system.

More specifically, the endoscope of each embodiment is, for example, an illustration of an endoscope that is formed with an extremely small diameter and intended to be used mainly under an underwater environment.

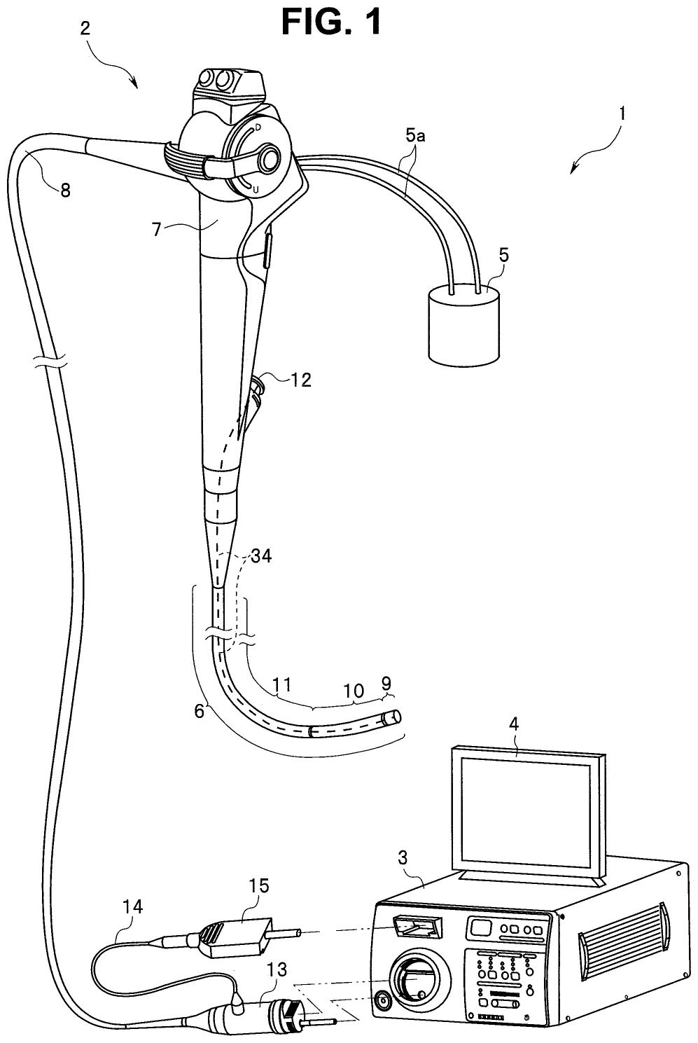

First, the following describes an overall schematic configuration of an endoscope system including an endoscope, which is the insertion apparatus according to each embodiment of the present disclosure, with reference to FIG. 1.

FIG. 1 is a schematic configuration diagram showing an endoscope system including an endoscope that is the insertion apparatus according to each embodiment of the present disclosure.

As shown in FIG. 1, an endoscope system 1 includes an endoscope 2, a video processor with a built-in illumination light source (hereinafter simply referred to as a video processor) 3 including a light source device and an endoscope image processing device, a display device 4, a liquid feeding and suction pump 5 and the like.

The endoscope 2 includes an insertion portion 6 in elongated tubular shape, an operation portion 7, a universal cord 8 and the like.

The insertion portion 6 is a component portion that is inserted into the body cavity of the subject.

The insertion portion 6 is a component member having flexibility as a whole and an elongated tubular shape.

The insertion portion 6 internally includes a signal transmission cable and optical fiber cable (not shown). In addition, the insertion portion 6 internally includes a plurality of conduits (for example, a treatment instrument insertion channel and suction conduit, and a liquid feeding conduit; the details are to be described below) that are inserted and assembled from a proximal end to a distal end through the insertion portion 6 in a longitudinal direction.

The insertion portion 6 includes, sequentially from the distal end side, a distal end portion 9 that is the distal end portion of the insertion portion, a bending portion 10, and a flexible tube portion 11.

Inside the distal end portion 9, an image pickup unit, an illumination unit, and the like (not shown) are provided.

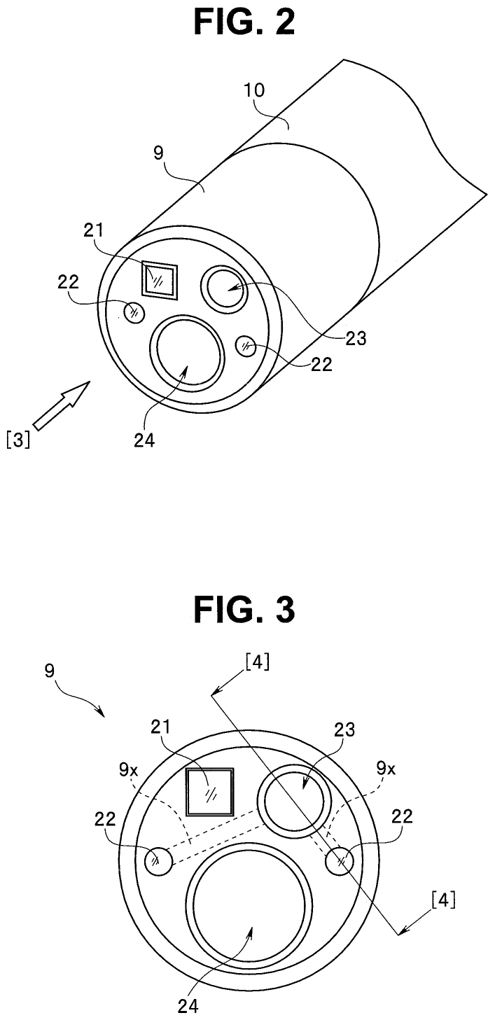

Therefore, as is to be shown below in FIG. 2, the distal end surface of the distal end portion 9 of the insertion portion 6 is provided with an observation window 21, illumination windows 22, a liquid feeding opening 23, a channel opening 24, and the like (details are to be described below). The insertion portion 6 includes a first conduit having a first opening, an optical fiber 32 configured to guide and radiate an illumination light. The first conduit is in fluid communication with an outside of the insertion portion via the first opening. The first conduit can be the suction conduit 34, the liquid feeding conduit 23 and other type of conduit. The suction conduit 34 can be used as the treatment instrument insertion channel. The first opening can be the liquid feeding opening 23, the suction opening and the channel opening 24. The first opening can be provided at a distal end surface of the insertion portion or a circumferential surface of the insertion portion. The first conduit is configured to suction a fluid through the first opening and toward a proximal end of the first conduit. The fluid can be a gas or a liquid, such as a bodily fluid, e.g., blood, digestive juice and residue.

The bending portion 10 can be bent freely by operating an operating member of the operation portion 7.

The flexible tube portion 11 is a long component member that has flexibility and an elongated tubular shape.

The operation portion 7 is connected to the proximal end of the flexible tube portion 11.

The operation portion 7 includes an operation portion body, a plurality of operation members, a forceps opening 12 and the like.

The operation portion body is box-shaped as a whole and configures a grasping portion.

As described above, the insertion portion 6 extends from the operation portion body.

The plurality of operation members are operating members through which various operations of the endoscope 2 is performed.

The plurality of operation members is provided at positions on the outer surface of the operation portion body.

The forceps opening 12 is provided at a position on the operation portion body of the operation portion 7.

The forceps opening 12 is a proximal end side opening to be used when a treatment instrument (not shown) having an elongated tubular shape is inserted through a treatment instrument insertion channel and suction conduit (see a reference numeral 34 in FIG. 1; details are to be described below) provided in the insertion portion 6 of the endoscope 2.

The universal cord 8 is a tubular member extending from a lateral side of the operation portion 7.

The distal end of the universal cord 8 is provided with a scope connector 13.

The scope connector 13 is connected to the video processor 3.

From the scope connector 13, optical cables 14 branch and extend.

The distal end of the optical cable 14 is provided with an optical connector 15.

The optical connector 15 is also connected to the video processor 3.

Here, the video processor 3 is a video processor with a built-in illumination light source including a light source device and an endoscope image processing device, as described above.

A light source device built in the video processor 3 supplies illumination light to an illumination unit (not shown in FIG. 1) provided inside the distal end portion 9 of the insertion portion 6 of the endoscope 2.

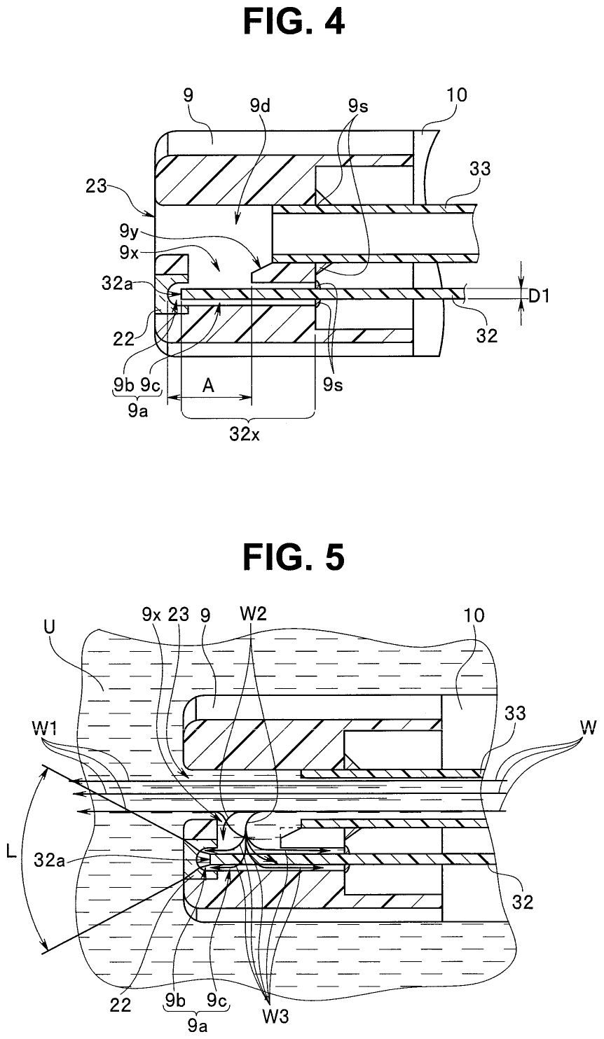

Illumination light emitted from the light source device of the video processor 3 is transmitted to the illumination unit in the distal end portion 9 of the insertion portion 6 of the endoscope 2 through an optical fiber cables or the like (not shown in FIG. 1; see a reference numeral 32 in FIG. 4; details are to be described below) that are placed to be inserted through the optical connector 15, the optical cable 14, the scope connector 13, the universal cord 8, the operation portion 7, and the insertion portion 6.

The illumination light passes through the illumination windows 22 (see FIG. 2) provided on the front surface of the distal end portion 9 and is radiated toward an object to be observed or treated in front of the distal end portion 9.

Also, the video processor 3 is a control device that controls the entire endoscope system 1.

In addition, the video processor 3 has a processing device configured to appropriately process endoscope images acquired by the image pickup unit of the endoscope 2.

For the purpose, the video processor 3 includes a signal processing circuit configured to receive image pickup signals from an image pickup unit (not shown) provided inside the distal end portion 9 of the insertion portion 6 of the endoscope 2 and performs signal processing.

The video processor 3 also includes a control processing circuit and the like configured to output control signals and the like to drive the image pickup unit.

The video processor 3 is electrically connected to the image pickup unit by signal transmission cables (not shown).

Therefore, the signal transmission cables are placed to be inserted through the scope connector 13, the universal cord 8, the operation portion 7, and the distal end portion 9 of the insertion portion 6.

With the configuration, image pickup signals outputted from the image pickup unit, control signals outputted from the video processor 3, and the like are transmitted between the image pickup unit and the video processor 3 through the signal transmission cables.

Note that one form of the signal transmission cables to be applied is, for example, a composite cable in which a plurality of cables is bundled and covered with an outer skin shield, an outer skin tube, or the like.

Also, the video processor 3 is connected to the display device 4 with video cables (not shown).

The video cables transmit image signals, control signals, and the like, which are outputted from the video processor 3, to the display device 4.

The display device 4 receives image signals and control signals outputted from the video processor 3, and displays an endoscope image and various information in a form according to a display mode in accordance with the received control signals.

The liquid feeding and suction pump 5 is a pump device including a liquid feeding pump for perfusion and a suction pump for suction.

The liquid feeding and suction pump 5 is a device configured to forcibly cause a liquid to flow between the body cavity and the outside.

For the purpose, the liquid feeding and suction pump 5 is disposed on the proximal end sides (the other end side) of a liquid feeding conduit (first conduit to be described below) and a suction conduit (second conduit to be described below), respectively.

The liquid feeding and suction pump 5 is connected to the operation portion 7 through a liquid feeding tube and a suction tube 5a.

Note that the liquid feeding pump is a pump device for perfusion configured to forcibly feeding a liquid such as physiological saline into the body cavity through a liquid feeding conduit (not shown in FIG. 1; details are to be described below) of the insertion portion 6.

The suction pump is a pump device for suction configured to suction an object to be suctioned such as a liquid in the body cavity (the liquid sent into the body cavity, blood caused by bleeding, etc.), to discharge the object out of the body cavity, through a treatment instrument insertion channel and suction conduit (see a reference numeral 34 in FIG. 1; details are to be described below) of the insertion portion 6.

Other configurations in the endoscope system 1 are the same as configurations of similar conventional endoscope systems.

Each embodiment of the endoscope included in the endoscope system 1 thus configured is to be described in detail below.

FIG. 2 is an enlarged perspective view of a main part showing an enlarged distal end portion of an insertion portion in an endoscope, which is an insertion apparatus of a first embodiment of the present disclosure.

FIG. 3 is a plan view of the distal end surface of the distal end portion in FIG. 2 as seen in the direction of an arrow [3] in FIG. 2.

FIG. 4 is a cross-sectional view taken along a line [4]-[4] in FIG. 3.

As described above and as shown in FIGS. 2 and 3, the distal end surface of the distal end portion 9 is provided with an observation window 21, illumination windows 22, a liquid feeding opening 23, a channel opening 24, and the like.

Note that the distal end portion 9 is made of a resin material such as polysulfone resin (hereinafter referred to as PSU resin).

At least part of the distal end portion 9 (for example, the portion where the observation window 21 and the illumination windows 22 are disposed) is configured to be transparent.

The observation window 21 is a light-transmitting optical member that configures part of an observation optical system (not shown).

The observation optical system is a component member having a function of forming an optical image of an object to be observed or treated.

Behind the observation optical system, an image pickup unit including an image pickup device, and a drive circuit and a signal processing circuit for the device and the like are disposed, which are not shown.

The illumination windows 22 are light-transmitting optical members that configure parts of the illumination unit.

Here, the illumination unit is an illumination portion that illuminates an object to be observed or treated in a body cavity.

The illumination unit includes illumination lenses and a light emitting portions.

Note that details of the illumination lenses and the light emitting portions included in the illumination unit are to be described below.

Each of the illumination windows 22 is an optical member that configures part of the illumination lens that passes illumination light, which is transmitted from the light source device of the video processor 3 to the distal end of the insertion portion 6 through an optical fiber cable 32 (optical fiber) (see FIG. 4), and radiates the illumination light toward an object to be observed or treated in front of the distal end portion 9.

For the purpose, the illumination window 22 has a shape such as a plano-concave lens shape that has a function of diverging light rays.

In other words, the illumination window 22 is formed to have a curved surface with a curvature on the inner surface side.

The insertion portion 6 includes the illumination lens includes the illumination window 22. The illumination window 22 is spaced apart from a distal end of the optical fiber 32 to form a space. The illumination chamber is configured to hold a liquid in the space. The illumination window 22 may have an optical property of concentration or dispersion of the light passing through the illumination window 22, or merely transparent the light passing through the illumination window 22.

Behind the illumination window 22, as shown in FIG. 4, the optical fiber cable 32 is disposed that is a light guide member configured to guide illumination light from the optical device.

For the purpose, the distal end portion 9 has an opening facing the proximal end surface of the distal end portion 9, and has a non-through hole 9a that extends toward the distal end side of the distal end portion 9 in the longitudinal direction.

Here, as described above, the optical fiber cable 32 is inserted through the insertion portion 6, the operation portion 7, the universal cord 8, the scope connector 13, the optical cable 14, and the optical connector 15, and is thereby disposed to be inserted through from the distal end portion 9 to the light source device of the video processor 3.

In the endoscope 2 according to the first embodiment, one in which the diameter D1 (see FIG. 4) of the optical fiber cable 32 is, for example, about 0.25 mm is applied.

The optical fiber cable 32 is placed in a state in which part of the optical fiber cable 32 on the distal end side is inserted into the non-through hole 9a.

At the time, as shown in FIG. 4, a gap 9c is provided between the lateral side surface of the optical fiber cable 32 and the inner surface of the lateral side of the non-through hole 9a.

Further, in a state in which part of the optical fiber cable 32 on the distal end side is inserted into the non-through hole 9a, the proximal end side opening of the non-through hole 9a is sealed in a watertight manner with, for example, an adhesive or the like 9s (See FIG. 4, etc.). The illumination chamber is connected to an optical fiber conduit, portions of the optical fiber 32 are located in the optical fiber conduit, and a gap between an inner surface of the optical fiber conduit and the optical fiber 32 is sealed to form a watertight structure.

Further, in a state in which part of the optical fiber cable 32 on the distal end side is inserted into the non-through hole 9a, the distal end surface 32a of the optical fiber cable 32 is placed at a position facing the inner surface (concave surface) of the illumination window 22.

Here, an area of the optical fiber cable 32, on the distal end side, including the distal end surface 32a of the optical fiber cable 32 is called a light emitting portion 32x.

More specifically, the light emitting portion 32x denotes part of the optical fiber cable 32 on the distal end side which part is inserted into the non-through hole 9a (see FIG. 4).

In the case, in a state in which part of the optical fiber cable 32 on the distal end side is inserted into the non-through hole 9a, a gap 9b is formed that has a interval between the distal end surface 32a of the optical fiber cable 32 and the inner surface (concave surface) of the illumination window 22.

With the configuration, illumination light emitted from the distal end surface 32a of the optical fiber cable 32 enters the inner surface of the illumination window 22 through the gap 9b.

Although details are to be described below, the endoscope 2 of the first embodiment has the gaps 9b and 9c filled with a liquid such as physiological saline in use of the endoscope 2.

At the time, the gap 9b functions as part of the illumination lens by being filled with a liquid.

Therefore, in the endoscope 2 of the first embodiment, the illumination lens is formed by the illumination window 22 and the gap 9b filled with a liquid.

In addition, the internal space of the non-through hole 9a in the distal end portion 9 is a space that includes the gap 9b that functions as part of the illumination lens and accommodates the light emitting portion 32x.

Therefore, in the following descriptions, the space is to be referred to as an illumination chamber.

Also, as described above, a watertight structure is formed such that the illumination chamber (the internal space of the non-through hole 9a) in the distal end portion 9 is sealed in a watertight manner with the adhesive or the like 9s against the outside on the proximal end side of the light emitting portion 32x. The insertion portion 6 includes the illumination chamber having a second opening 9x. The illumination chamber is configured to fluidly communicate with a peripheral part of the first conduit via the second opening 9x. A distal end of the optical fiber is located inside of the illumination chamber. The illumination lens is configured to radiate the illumination light to the outside of the insertion portion.

Note that, the endoscope 2 of the first embodiment illustrates a configuration example which has the illumination windows 22 provided at positions opposing each other with the observation window 21 interposed between the illumination windows 22.

The liquid feeding opening 23 is a distal end opening of a liquid feeding conduit (not shown in FIG. 1; see a reference numeral 33 in FIG. 4), which is the first conduit inserted through the insertion portion 6.

Here, the liquid feeding conduit is a component portion that extends rearward from the liquid feeding opening 23 and is formed in a tubular shape.

The liquid feeding conduit is a flow path for perfusing the front of the distal end portion 9 of the insertion portion 6 with a liquid such as physiological saline fed from the outside.

In other words, the distal end portion 9 has a liquid feeding path through hole 9d behind the liquid feeding opening 23. The liquid feeding path through hole 9d configures part of the liquid feeding conduit.

The distal end of a liquid feeding tube 33, which is a tubular member, is fixed to the proximal end side of the liquid feeding path through hole 9d.

In the case, the liquid feeding tube 33 is fixed to the distal end portion 9 with the adhesive or the like 9s.

Here, although illustration is omitted, the liquid feeding tube 33 is disposed so as to be inserted through the insides of the insertion portion 6 and the operation portion 7.

With such a configuration, the endoscope 2 of the first embodiment has the liquid feeding conduit formed by the liquid feeding path through hole 9d of the distal end portion 9 and the liquid feeding tube 33.

In other words, the liquid feeding conduit in the endoscope 2 is a conduit inserted through the insertion portion 6.

The liquid feeding conduit functions as a conduit that allows a liquid such as physiological saline to flow between one end side of the conduit and the other end side of the conduit in a state in which the insertion portion 6 is inserted into the body cavity of the subject in use of the endoscope 2.

More specifically, when the insertion portion 6 is inserted into the body cavity of the subject in use of the endoscope 2, the liquid feeding conduit functions as a conduit configured to allow a liquid such as physiological saline to flow between the inside of the subject where the distal end side of the conduit is disposed and the outside of the insertion portion 6 where the proximal end side of the conduit is disposed (liquid feeding pump).

In still other words, in a state in which the insertion portion 6 is inserted into the body cavity of the subject in use of the endoscope 2, the liquid feeding conduit functions as a first conduit configured to allow a liquid such as physiological saline to flow towards a part where one end side of the conduit (distal end side of the conduit) is disposed (the part inside the subject) from a part where the other end side of the conduit (proximal end side of the conduit) is disposed (the part outside the insertion portion 6 leading to the liquid feeding pump).

The channel opening 24 serves as both the distal end side opening of the treatment instrument insertion channel and the distal end side opening of the suction conduit.

Here, the treatment instrument insertion channel is a conduit that allows insertion of a treatment instrument (not shown) from the outside of the insertion portion 6 toward the inside of the subject.

Also, the suction conduit is a conduit to be used to suction an object to be suctioned in the body cavity to discharge the object outside.

In other words, in the endoscope 2 of the first embodiment, a single conduit is configured to be used for both the treatment instrument insertion channel and the suction conduit.

Therefore, in the following descriptions, the treatment instrument insertion channel and suction conduit (see a reference numeral 34 in FIG. 1) is simply to be referred to as a suction conduit.

The suction conduit is provided as a second conduit that is different from the above-described first conduit (liquid feeding conduit).

Simply saying, the suction conduit in the endoscope 2 is a conduit inserted through the insertion portion 6.

In a state in which the insertion portion 6 is inserted into the body cavity of the subject in use of the endoscope 2, the suction conduit functions as a conduit configured to allow an object to be suctioned such as a liquid to flow between a part where one end side of the conduit (distal end side of the conduit) is disposed (a part inside the subject) and a part where the other end side of the conduit (proximal end side of the conduit) is disposed (a part outside the insertion portion 6 leading to the suction pump).

In other words, in a state in which the insertion portion 6 is inserted into the body cavity of the subject in use of the endoscope 2, the suction conduit functions as a conduit configured to allow an object to be suctioned such as a liquid to flow from a part where the one end side of the conduit (the distal end side of the conduit) is disposed (a part inside the object) towards a part where the other end side of the conduit (proximal end side of the conduit) is disposed (a part outside the insertion portion 6 leading to the suction pump).

The suction conduit is provided as the second conduit different from the first conduit (liquid feeding conduit or discharge conduit).

The suction conduit, which is the second conduit, is formed to have a larger inner diameter than the liquid feeding conduit, which is the first conduit. The discharge conduit has a discharge opening, the discharge conduit is in fluid communication with the outside of the insertion portion 6 via the discharge opening. The suction conduit has a first inner diameter, the discharge conduit has a second inner diameter, and the second inner diameter is larger than the first inner diameter.

A suction conduit extends behind the channel opening 24.

The suction conduit is disposed to be inserted through the insides of the insertion portion 6 and the operation portion 7 in the same manner as the liquid feeding conduit described above.

Inside the operation portion 7, the suction conduit branches into a conduit leading to the forceps opening 12 and a conduit leading to a connection opening (not shown) of a suction tube.

Note that, in the first embodiment, the suction conduit is a portion that is not directly related to the present disclosure, so the illustration is omitted and further description is omitted.

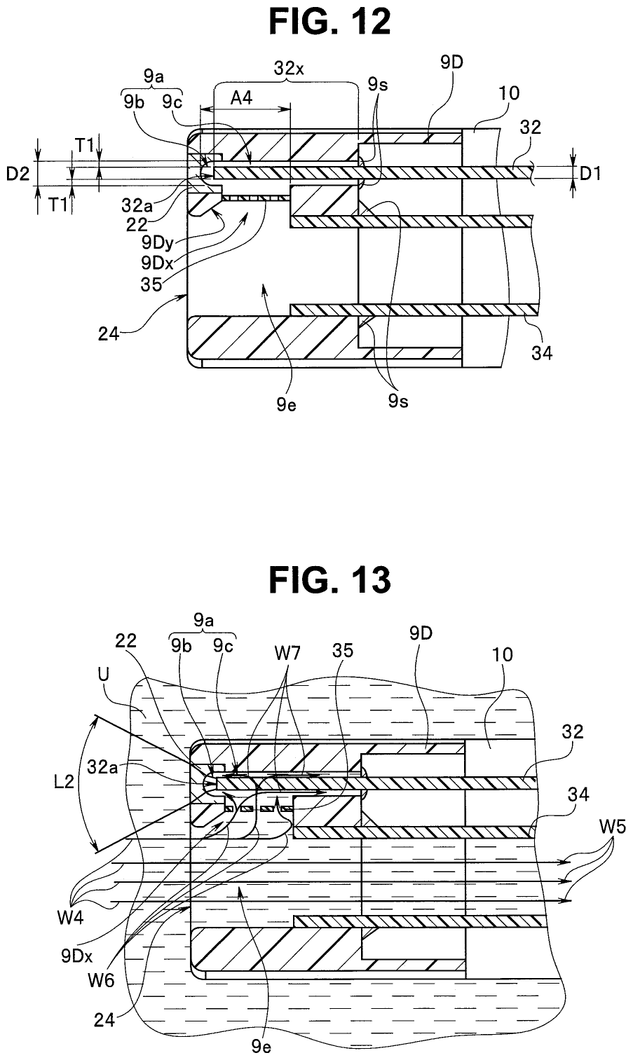

However, the part is illustrated and described in detail with reference to FIG. 12 and so on according to a fourth embodiment to be described below.

The endoscope 2 of the first embodiment has flow paths 9x formed at a site inside the distal end portion 9.

Each of the flow paths 9x is a flow path configured to allow a liquid to flow through the liquid feeding opening 23 on one end side (distal end side) of the liquid feeding path through hole 9d forming part of the liquid feeding conduit, the inside of the liquid feeding conduit (liquid feeding path through hole 9d) and the illumination chamber (internal space of the non-through hole 9a).

In other words, the flow path 9x is formed to be a path inside the distal end portion 9 that allows the liquid feeding path through hole 9d (liquid feeding conduit) to communicate with the inside of the illumination chamber (non-through hole 9a) to allow the liquid to flow through the liquid feeding path through hole 9d and the illumination chamber.

With the configuration, the flow path 9x has the function of allowing the liquid to flow through the inside of the illumination chamber (especially the inside of the gap 9b functioning as part of the illumination lens).

Note that, in the endoscope 2 of the first embodiment, the flow path 9x is configured to be connected only to the liquid feeding conduit (first conduit) inside the distal end portion 9.

In the case, the flow path 9x can be disposed in an area (see a reference character A in FIG. 4) within 3 mm from the illumination lens (the inner surface of the illumination window 22) in the longitudinal axis direction of the distal end portion 9.

Here, the area indicated by the reference character A in FIG. 4 is called a flow path area, for example.

The reason why the flow path area A is thus set to 3 mm or less is as follows.

Also, a slope (guide part) 9y is provided at a position in the boundary portion between the flow path 9x and the liquid feeding conduit (first conduit).

The slope 9y is provided in the vicinity of the boundary portion where the flow path 9x is connected to the distal end portion of the liquid feeding tube 33 that forms the liquid feeding conduit (first conduit).

In other words, the slope 9y is formed at a position closer to the proximal end in the flow path area A. The first conduit includes the guide part 9y configured to guide a liquid from the first conduit toward the illumination chamber. The guide part 9y has an inclined surface provided around at least a portion of a periphery of the second opening. The guide part 9y is provided proximally relative to the second opening 9x. In some cases, the inclined surface is oriented relative to a longitudinal axis of the insertion portion at a non-90 degree angle, for example, 5 to 50 degrees and in other instances the inclined surface is oriented relative to a longitudinal axis of the insertion portion at a 90 degree angle.

Here, the slope 9y is formed as an inclination that expands the liquid feeding conduit from the proximal end side toward the distal end side.

The slope 9y formed in such a manner causes part of the liquid feeding conduit to enter part of the flow path 9x.

With the configuration, the slope 9y changes the flow path of part of the liquid flowing toward the distal end side in the liquid feeding conduit, thereby facilitating the flow of the liquid into the flow path 9x.

In the case, the slope 9y functions as a flow path changing portion.

The slope 9y provided in such a way forms a structure in which the liquid flowing through the liquid feeding conduit flows more easily on the upstream side of the conduit than on the downstream side of the conduit.

The following describes actions of illuminating the inside of a body cavity of the subject with the endoscope 2, which is the insertion apparatus of the first embodiment configured in the above-described manner.

Note that FIG. 5 is a diagram for explaining the actions of the endoscope according to the first embodiment of the present disclosure in illuminating the inside of a body cavity of the subject.

Here, FIG. 5 shows a cross section of the distal end portion located in the body cavity.

First, the insertion portion 6 of the endoscope 2 is inserted into the body cavity of the subject according to normal procedures (not shown). Then, the distal end portion 9 of the insertion portion 6 is placed in the vicinity of a target site to be observed or treated (not shown). When the distal end portion 9 of the insertion portion 6 thus reaches the vicinity of the site to be observed or treated, the liquid feeding pump in the liquid feeding and suction pump 5 is actuated.

Then, the liquid feeding and suction pump 5 starts perfusion to send a liquid such as physiological saline from the outside of the insertion portion 6 into the body cavity of the subject through the liquid feeding conduit.

In FIG. 5, an arrow mark W conceptually indicates the direction of flow of the liquid flowing through the liquid feeding conduit toward the inside of the body cavity.

At the time, when the liquid flowing toward the distal end of the liquid feeding conduit reaches the distal end portion 9, part of the liquid flows into the body cavity from the liquid feeding opening 23 on the distal end surface.

In FIG. 5, an arrow mark W1 conceptually indicates the flow direction of the liquid discharged from the liquid feeding opening 23 into the body cavity.

A reference character U in FIG. 5 indicates how the body cavity is filled with the liquid that has flowed into the body cavity from the liquid feeding opening 23.

At the time, the other part of the liquid that has reached the distal end portion 9 flows into the flow path 9x.

In FIG. 5, an arrow mark W2 conceptually indicates the flow direction of the liquid flowing into the flow path 9x.

Then, the liquid that has flowed into the flow path 9x flows into the illumination chambers (non-through holes 9a).

In FIG. 5, an arrow mark W3 conceptually indicates the flow direction of the liquid flowing from the flow path 9x into the illumination chamber (non-through hole 9a).

As a result, the inside of the illumination chamber (non-through hole 9a) is filled with the liquid.

After the illumination chamber (non-through hole 9a) is thus filled with the liquid, the light source device of the video processor 3 is operated and the illumination unit is actuated.

Thereby, illumination light is emitted from the light source device.

Illumination light emitted from the light source device reaches the illumination unit through the optical fiber cables 32.

Then, the illumination light is emitted forward from the distal end surfaces 32a of the light emitting portions 32x.

At the time, the illumination light passes through the gap 9b (illumination lens) and the illumination window 22 and is emitted forward from the distal end portion 9.

Thereby, the illumination light is radiated toward the object to be observed or treated in the body cavity of the subject.

A reference character L in FIG. 5 conceptually indicates the irradiation range of the illumination light, which is emitted from the distal end surface 32a of the light emitting portion 32x, passes through the illumination lens (the gap 9b filled with liquid and the illumination window 22) and is radiated forward from the distal end portion 9.

In the case, the shape of the illumination lens (a curvature of the inner surface of the illumination window 22) is determined to exhibit higher light distribution characteristics in a case in which the medium (a liquid such as physiological saline) filled in the gap 9b (part of the illumination lens) has a refractive index of 1.33 and the material of the illumination window 22 (part of the illumination lens) has a refractive index of 1.63. the illumination lens includes an illumination window extending from the distal end surface of the insertion portion 6 to a distal end surface of the illumination chamber.

As described above, according to the first embodiment, the endoscope 2 capable of perfusion or suction through a conduit and to be used under an underwater environment has a configuration such that flow paths 9x are provided to allow the liquid feeding conduit to communicate with the illumination chambers to allow the liquid to flow through the liquid feeding conduit and the illumination chambers.

With the configuration, when liquid is fed, part of the liquid flowing toward the distal end portion 9 through the liquid feeding conduit is discharged forward from the distal end portion 9.

At the same time, part of the liquid can flow into each of the flow paths 9x and fill the gap 9b portions in each of the illumination chambers with the liquid.

In the case, during liquid feeding, the gap 9b portions are maintained to be filled with a new liquid.

Moreover, since the slope 9y is provided at a position in the boundary portion between the flow path 9x and the liquid feeding conduit, the liquid flowing from the liquid feeding conduit can be smoothly introduced into the flow path 9x.

Thus, in the endoscope 2 of the first embodiment, a configuration is achieved such that the liquid is actively introduced into the gap 9b portion in the illumination chamber so that the gap 9b and the illumination window 22 function as an illumination lens.

Then, the configuration is determined so that appropriate light distribution characteristics are exhibited in a state in which a liquid is introduced into the gaps 9b.

Therefore, even in the endoscope 2 used under an underwater environment, there is no need to consider the difficulty of ensuring watertightness due to downsizing and reduction in the diameter.

The simple configuration in which the flow path 9x is provided, can provide stable illumination light distribution characteristics.

Next, an endoscope of a second embodiment of the present disclosure is to be explained below.

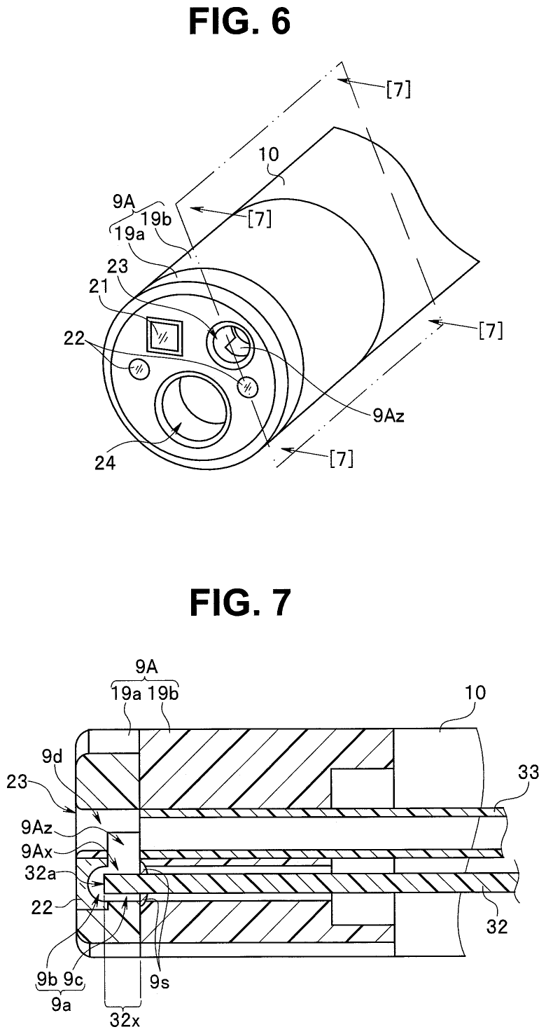

FIG. 6 is an enlarged perspective view of a main part showing an enlarged distal end portion of an insertion portion in an endoscope, which is an insertion apparatus according to the second embodiment of the present disclosure.

FIG. 7 is a cross-sectional view of the distal end portion of FIG. 6 taken along the plane indicated by arrows [7] in FIG. 6.

Note that the cross section taken along the plane indicated by arrows [7] in FIG. 6 corresponds to the cross section taken along the line [4]-[4] in FIG. 3.

The basic components of a distal end portion (distal end portion 9A) of the insertion portion in the endoscope of the second embodiment of the present disclosure are the same as the components of the first embodiment described above.

The second embodiment differs in that the distal end portion 9A is configured with two components.

Therefore, the same reference numerals and characters are to be given and the descriptions are to be omitted for the same components as the components of the above-described first embodiment, and the descriptions are to be made below for the different components.

In the endoscope of the second embodiment, the distal end portion 9A is configured with two components: a distal end portion cover 19a that is a first distal end portion; and a distal end portion body 19b that is a second distal end portion.

These two components (19a and 19b) are integrally connected in the longitudinal direction of the distal end portion 9A.

In the case, the two components (19a and 19b) are adhered and bonded in a watertight manner.

The distal end portion cover 19a is disposed on the distal end side of the distal end portion 9A.

At least part of the distal end portion cover 19a is transparent.

For example, the portion where the observation window 21 and the illumination windows 22 are disposed is made transparent.

The distal end portion cover 19a has illumination chambers.

Here, each of the illumination chambers is a space that includes the gap 9b that functions as part of the illumination lens and accommodates the light emitting portion 32x, as in the first embodiment described above.

In other words, the illumination chamber corresponds to the internal space of the non-through hole 9a.

Also, the distal end portion cover 19a is integrally formed with at least a portion that functions as an illumination lens.

Here, the portion functioning as an illumination lens corresponds to the illumination window 22 and the gap 9b.

The distal end portion cover 19a is made of, for example, PSU resin.

Furthermore, the distal end portion cover 19a has flow paths 9Ax.

As in the first embodiment described above, each of the flow paths 9Ax is a flow path configured to allow a liquid to flow through the liquid feeding opening 23 on the distal end side of the liquid feeding path through hole 9d, which is part of the liquid feeding conduit, the inside of the liquid feeding path through hole 9d, and the illumination chamber (internal space of the non-through hole 9a). The illumination chamber is configured to fluidly communicate with the peripheral part of the liquid feeding conduit via the second opening 9Ax.

Note that, in the second embodiment, inside the distal end portion cover 19a, the flow path 9Ax is provided with a flow path to allow the liquid feeding conduit to communicate with the inside of the illumination chamber to allow the liquid to flow through the liquid feeding conduit and the illumination chamber. In addition, the flow path 9Ax is provided with a cut-out portion (guide part) 9Az formed by expanding part of the liquid feeding path through hole 9d, which is part of the liquid feeding conduit.

The cut-out portion 9Az is a flow path changing portion provided in place of the slope 9y in the above-described embodiment.

With such a configuration, the cut-out portion 9Az changes the flow path of part of the liquid flowing toward the distal end through the liquid feeding conduit, thereby making it easier for the liquid to flow into the flow path 9Ax.

The first conduit includes the guide part 9Az configured to guide a liquid from the liquid feeding conduit toward the illumination chamber. The guide part 9Az has a recessed surface may be provided distally relative to the second opening 9Ax, the recessed surface is recessed radially.

The distal end portion body 19b is connected to the distal end portion cover 19a (first distal end portion) and disposed on the proximal end side of the distal end portion 9A.

The distal end portion body 19b is configured as a component separate from the distal end portion cover 19a (first distal end portion).

The distal end portion body 19b is made of, for example, a resin material.

The optical fiber cable 32 and the liquid feeding tube 33 are disposed to be inserted through the distal end portion body 19b.

For the purpose, through holes through which the tubular members are inserted is formed in the distal end portion body 19b in the axial direction of the distal end portion 9.

In addition, an image pickup unit (not shown) and the like are disposed in the distal end portion body 19b.

Further, the distal end portion body 19b has a watertight structure, on the proximal end side of the light emitting portion 32x, to make the connection with the inside of the illumination chamber watertight, that is, to make the portion corresponding to the gap 9c watertight.

For the purpose, the opening on the proximal end side of the non-through hole 9a is sealed with, for example, the adhesive 9s or the like in a watertight manner.

Other configurations and actions in the distal end portion 9A are the same as the configurations and actions of the above-described first embodiment.

As described above, according to the second embodiment, it is possible to obtain the same actions and effects as the actions and effects of the first embodiment.

Furthermore, according to the second embodiment, the distal end portion 9A is configured with the two components (19a and 19b) so that the flow paths 9Ax and the flow path changing portions 9Az can be formed more easily in the manufacturing process.

In addition, in the second embodiment, the distal end portion 9A is configured with the two components (19a and 19b), and the two components are adhered and bonded in a watertight manner.

In the case, the liquid feeding tube 33 can have both side surfaces bonded to the through hole of the distal end portion body 19b (second distal end portion) in a state in which the liquid feeding tube 33 is inserted through the through hole.

Such a configuration allows the liquid feeding tube 33 to be more reliably fixed to the distal end portion 9A. At the same time, a watertight structure around the liquid feeding tube 33 can be achieved more easily.

Furthermore, the watertight structure of each of the illumination chambers can be easily formed simply by sealing the opening portion of the non-through hole 9a on the proximal side (the proximal end side of the illumination chamber) in a watertight manner.

Therefore, according to the present configuration, it is possible to contribute to the simplification of the manufacturing process, thereby contributing to the reduction of the manufacturing cost.

Next, an endoscope of a third embodiment of the present disclosure is to be explained below.

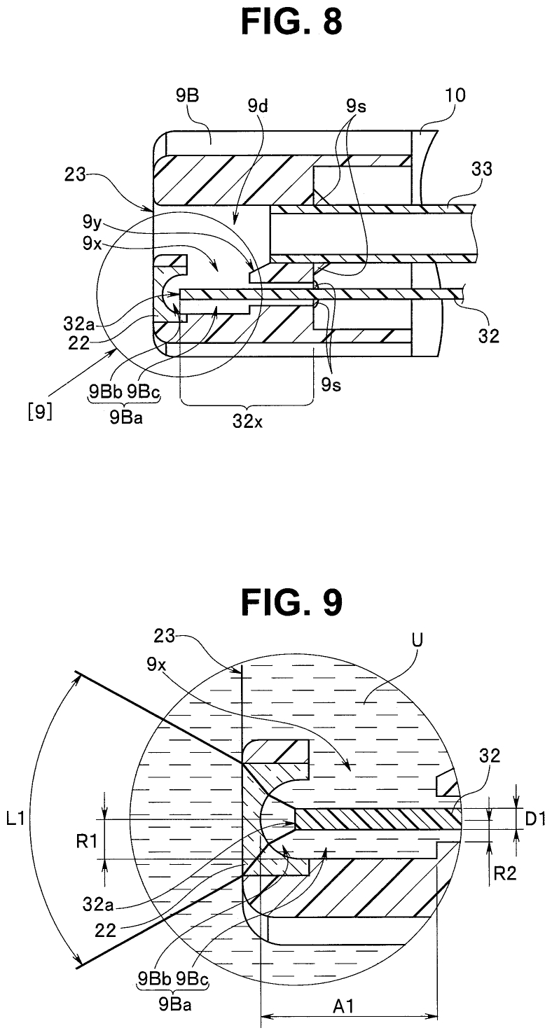

FIG. 8 is a cross-sectional view showing an enlarged distal end portion of an insertion portion in the endoscope, which is an insertion apparatus according to the third embodiment of the present disclosure.

Note that FIG. 8 corresponds to a cross section taken along line [4]-[4] in FIG. 3, as in the first embodiment. FIG. 9 is an enlarged cross-sectional view of a main part showing an enlarged cut-out area indicated by an arrow mark [9] in FIG. 8.

The basic components of the distal end portion of the insertion portion (distal end portion 9B) in the endoscope of the third embodiment of the present disclosure are the same as the components of the first embodiment described above.

The third embodiment differs in the forms of portions that function as illumination lenses in the illumination chambers formed inside the distal end portion 9B.

Therefore, the same reference numerals and characters are to be given and the descriptions are to be omitted for the same components as the components of the above-described first embodiment, and the descriptions are to be made below for the different components.

In the endoscope of the third embodiment, the distal end portion 9B has portions of the illumination chambers (non-through holes 9Ba), each of which portions includes a gap 9Bb and corresponds to the flow path 9x (a flow path area indicated by a reference character A1 in FIG. 9). Each of the portions has a diameter set larger than the diameter in the first embodiment.

More specifically, each of the flow path areas A1 in the illumination chambers (non-through holes 9Ba) has a radius (see a reference character R1 in FIG. 9) set to twice or more the diameter D1 of the optical fiber cable 32 (R1≥D1×2).

Also, in the case, the illumination chamber (non-through hole 9Ba) has a radius in the area on the proximal end side of the flow path area A1 (see a reference character R2 in FIG. 9) that is set smaller than the radius R1 of the flow path area A1 (R1>R2).

Other configurations and actions are the same as the configurations and actions of the above-described first embodiment.

As described above, according to the third embodiment, the same effects as effects of the first embodiment can be obtained.

Furthermore, according to the third embodiment, the diameter of the gap 9Bb in the illumination chamber (non-through hole 9Ba) can be increased.

The configuration makes it possible that the illumination window 22B has an increased diameter and an increased radius of the curvature of the concave surface formed on the inner surface.

As the illumination window 22B has a greater radius of the curvature of the inner surface (concave surface), the light distribution characteristics are given a greater degree of freedom.

Therefore, it is possible to obtain illumination light distribution characteristics that allow irradiation in a larger range (see a reference character L1 in FIG. 9).

In addition, since the diameter R2 of the area on the proximal end side is set smaller than the diameter R1 of the flow path area A1 (R1>R2), the ease of forming the watertight structure is maintained.

Therefore, the watertight structure of the illumination chamber can be reliably ensured.

Note that the configuration of the distal end portion of the third embodiment described above can also be applied to the distal end portion of the second embodiment described above.

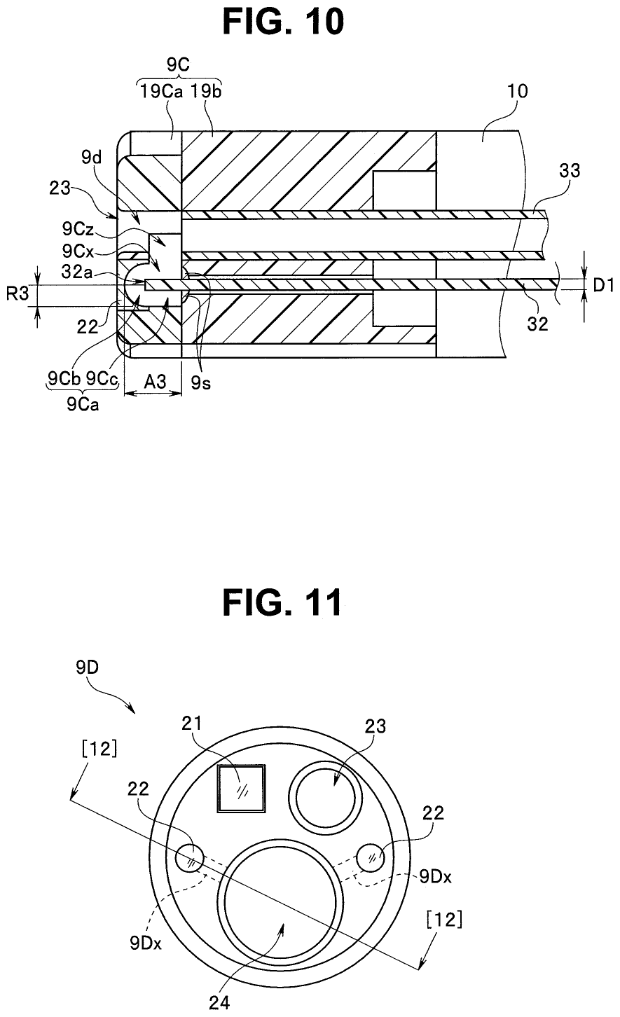

FIG. 10 is a diagram showing a modification of the third embodiment of the present disclosure.

In other words, FIG. 10 shows a cross section of the distal end portion of the modification of the third embodiment, and is a cross-sectional view corresponding to FIGS. 4, 7, and 8.

The modification shown in FIG. 10 illustrates a case in which the same configuration (illumination chamber with a larger diameter) of the distal end portion in the third embodiment described above is applied to the configuration of the distal end portion in the second embodiment (distal end portion configured with the two components) described above.

As shown in FIG. 10, the distal end portion 9C of the modification is configured with two components: a distal end portion cover 19Ca that is a first distal end portion; and a distal end portion body 19b that is a second distal end portion.

These two components (19Ca and 19b) are connected in the longitudinal direction of the distal end portion 9C to be integrated in a watertight manner. The illumination chamber is configured to fluidly communicate with the peripheral part of the first conduit via the second opening 9Cx.

The diameter of a flow path area A3 of each of the illumination chambers (non-through hole 9Ca) formed in the distal end portion cover 19Ca is increased as in the third embodiment.

More specifically, the radius R3 of the flow path area A3 of the illumination chamber (non-through hole 9Ca) is set to twice or more the diameter D1 of the optical fiber cable 32 (R3≥D1×2).

Other configurations and actions are the same as the configurations and actions of the above-described second embodiment.

According to such a modification in the configuration, the distal end portion 9C configured with the two components (19Ca and 19b) enables obtaining the same effects as the above-described second embodiment. At the same time, the configuration in which the illumination chamber has an increased diameter also enables obtaining the same effects as the above-described third embodiment.

In the first to third embodiments described above, each example of the configuration of the distal end portion is shown in which the flow paths is provided each to allow the liquid feeding conduit to communicate with the illumination chamber to allow the liquid to flow through the liquid feeding conduit and the illumination chamber.

In a fourth embodiment shown below, a configuration of the distal end portion is illustrated in which, instead of the liquid feeding conduit, flow paths are provided between the suction conduit and the illumination chambers to allow the liquid flow through the suction conduit and the illumination chambers.

An endoscope according to the fourth embodiment of the present disclosure is to be described below with FIGS. 11 to 13.

FIG. 11 is a plan view of the distal end surface of the distal end portion of the insertion portion in the endoscope, which is an insertion apparatus of the fourth embodiment of the present disclosure.

FIG. 12 is a cross-sectional view taken along a line [12]-[12] in FIG. 11. FIG. 13 is a diagram for explaining the action when the endoscope of the fourth embodiment of the present disclosure illuminates the inside of the body cavity of the subject.

Here, FIG. 13 shows a cross section of the distal end portion in a body cavity.

The basic components of the distal end portion of the insertion portion (distal end portion 9D) in the endoscope of the fourth embodiment of the present disclosure are substantially the same as the components of the first embodiment described above.

The fourth embodiment differs in the placement of flow paths 9Dx formed inside the distal end portion 9D.

Therefore, the same reference numerals and characters are to be given and the descriptions are to be omitted for the same components as the components of the above-described first embodiment, and the descriptions are to be made below for the different components.

As shown in FIG. 11, an observation window 21, illumination windows 22, a liquid feeding opening 23, a channel opening 24, and the like are provided on the distal end surface of the distal end portion 9D in the endoscope of the fourth embodiment.

Among the windows and openings, the observation window 21, the illumination windows 22, and the liquid feeding opening 23 are configured in the same manner as the ones in the above-described first embodiment.

Also, the channel opening 24 is the same as the above-described first embodiment in that the channel opening 24 is the distal end side opening of the treatment instrument insertion channel and also serves as the distal side opening of the suction conduit.

In other words, also in the endoscope according to the fourth embodiment, a single conduit is configured to be used for both the treatment instrument insertion channel and the suction conduit.

The suction conduit is provided as a second conduit different from the liquid feeding conduit (first conduit).

Here, the suction conduit is a component portion that extends rearward from the channel opening 24 and is formed in a tubular shape.

The suction conduit is a flow path configured to suction the liquid in the body cavity to discharge the liquid out of the insertion portion when the insertion portion is in a state of being inserted into the body cavity of the subject in use of the endoscope.

In other words, in the distal end portion 9D, a suction path through hole 9e is formed that configures part of the suction conduit behind the channel opening 24.

The suction path through hole 9e has the proximal end side fixed to the distal end of the treatment instrument insertion channel and suction tube 34 (hereinafter simply referred to as a suction tube) that is a tubular member and configures a suction conduit.

In the case, the suction tube 34 is fixed to the distal end portion 9D with the adhesive or the like 9s.

Note that, although illustration is omitted, the suction tube 34 is disposed to be inserted through the inside of the insertion portion and the operation portion.

With such a configuration, the endoscope of the fourth embodiment has the suction path through hole 9e of the distal end portion 9D and the suction tube 34 that form a suction conduit.

The inner diameter of the suction conduit (second conduit) is larger than the inner diameter of the liquid feeding conduit (first conduit).

Further, flow paths 9Dx are formed at sites inside the distal end portion 9D.

Each of the flow paths 9Dx is a flow path to allow a liquid to flow through: the channel opening 24 on one end side (distal end side) of the suction path through hole 9e forming part of the suction conduit; the inside of the suction conduit (suction path through hole 9e); and each of the illumination chambers (internal space of the non-through hole 9a). The illumination chamber is configured to fluidly communicate with the peripheral part of the suction conduit via the second opening 9Dx.

In other words, the flow path 9Dx is formed as a path configured to allow the suction path through hole 9e (suction conduit) to communicate with the inside of the illumination chamber (non-through hole 9a) inside the distal end portion 9D to allow the liquid to flow through the suction path through hole 9e and the inside of the illumination chamber.

With the configuration, the flow path 9Dx has the function of allowing the liquid to flow through the gap 9b, functioning as part of the illumination lens in the illumination chamber, and the gap 9c in the illumination chamber.

Note that, in the fourth embodiment, the flow path 9Dx is connected only to the suction conduit (second conduit) inside the distal end portion 9D.

In the case, the flow path 9Dx can be disposed in a flow path area A4, for example, within 3 mm from the illumination lens (the inner surface of the illumination window 22) in the longitudinal axis direction of the distal end portion 9D, as in the first embodiment described above.

In addition, a slope 9Dy that is a flow path changing portion is provided at a position in the boundary portion between the flow path 9Dx and the suction conduit (second conduit).

The slope 9Dy is provided in the vicinity of the boundary portion where the flow path 9Dx connects to the suction path through hole 9e forming part of the suction conduit (second conduit).

In other words, the slope 9Dy is formed at a position closer to the distal end in the flow path area A4.

Here, the slope 9Dy is formed as an inclination that expands the suction conduit (suction path through hole 9e) from the distal end side toward the proximal end side.

The formation of the slope 9Dy in such a manner causes part of the suction conduit to enter part of the flow path 9Dx.

With the configuration, the slope 9Dy changes the flow path of part of the liquid flowing toward the proximal end side in the suction conduit, thereby facilitating the flow of the liquid into the flow path 9Dx.

The slope 9Dy provided in such a way forms a structure in which the liquid flowing through the suction conduit flows more easily on the upstream side of the conduit than on the downstream side of the conduit. The first conduit includes the guide part 9Dy configured to guide a fluid from the first conduit toward the illumination chamber. The guide part 9Dy has an inclined surface provided around at least a portion of a periphery of the second opening. The guide part 9Dy is provided proximally relative to the second opening 9Dx.

In addition, a filter 35 is provided between each of the flow paths 9Dx in the fourth embodiment and the suction conduit (suction path through hole 9e) that is the second conduit.

The filter 35 prevents impurities and the like contained in an object to be suctioned, such as a liquid in the body cavity, from entering the illumination chamber (non-through hole 9a), the gap 9b, during suction.

Furthermore, considering the presence of impurities and the like contained in the object to be suctioned such as a liquid in the body cavity, the illumination chamber can have the gap 9c with the interval T1 (see FIG. 12) set to 0.1 mm or less.

Note that, for example, in a case in which the inside of an organ in the urinary system is the object to be observed or treated, the impurities and the like are supposed to be fragments of crushed calculus and the like. The illumination lens includes the illumination window 22. The illumination window 22 extends from a distal end surface of the insertion portion 6 to a distal end surface of the illumination chamber. A maximum distance between the distal end of the optical fiber 32 and a proximal end of the illumination window 22 is 0.1 mm.

With the setting, for example, if the diameter D1 of the optical fiber cable 32 is set to about 0.25 mm and the interval T1 of the gap 9c is set to 0.1 mm or less, a diameter D2 (see FIG. 12) of the non-through hole 9a is set to about 0.45 mm.

The following describes actions of illuminating the inside of the body cavity of the subject with the endoscope, which is the insertion apparatus of the fourth embodiment configured in the above-described manner with reference to FIG. 13 and the like.

When the distal end portion 9D of the insertion portion of the endoscope is inside the body cavity, the suction pump in the liquid feeding and suction pump 5 is actuated.

Then, the liquid feeding and suction pump 5 starts suctioning the object to be suctioned, such as a liquid in the body cavity, to discharge the object out of the insertion portion, through the suction conduit.

As a result, the liquid filling the body cavity (see a reference character U in FIG. 13) is suctioned from the channel opening 24 toward the suction conduit.

In FIG. 13, the reference character U indicates how the body cavity is filled with the liquid.

In FIG. 13, an arrow mark W4 conceptually indicates the direction in which an object to be suctioned, such as a liquid in the body cavity, flows from the channel opening 24 toward the suction conduit.

At the time, part of the liquid flowing from the channel opening 24 to the suction conduit passes through the suction path through hole 9e of the distal end portion 9D and flows through the suction conduit.

The part of the fluid is then suctioned out in the direction of the suction pump outside the insertion portion.

In FIG. 13, an arrow mark W5 conceptually indicates the flow direction of the liquid that flows through the suction conduit and is suctioned out in the direction of the suction pump.

Also, at the time, the other part of the liquid flowing from the channel opening 24 toward the suction conduit flows into the flow path 9Dx.

In FIG. 13, an arrow mark W6 conceptually indicates the direction in which the liquid flowing into the flow path 9Dx flows.

Then, the liquid that has flowed into the flow path 9Dx flows into the illumination chamber (non-through hole 9a).

At the time, impurities and the like contained in the object to be suctioned are prevented from flowing into the illumination chamber by the filter 35.

In FIG. 13, the arrow mark W7 conceptually indicates the flow direction of the liquid flowing from the flow path 9Dx into the illumination chamber (non-through hole 9a).

As a result, the inside of the illumination chamber (non-through hole 9a) is filled with the liquid.

After the illumination chamber (non-through hole 9a) is filled with the liquid in such a way, the illumination unit is actuated.

Then, the illumination light is emitted forward from the distal end surface 32a of the light emitting portion 32x.

At the time, the illumination light passes through the gap 9b (illumination lens) and the illumination window 22, and is radiated toward the object to be observed or treated in front of the distal end portion 9D.

A reference character L2 in FIG. 13 conceptually indicates an irradiation range of illumination light emitted from the distal end surface 32a of the light emitting portion 32x and passing through the illumination lens (the gap 9b filled with the liquid and the illumination window 22).

Then, the illumination light is radiated forward from the distal end portion 9D.

As described above, according to the fourth embodiment, the endoscope capable of perfusion or suction through a conduit and to be used under an underwater environment is provided with flow paths 9Dx each configured to allow the suction conduit to communicate with the illumination chamber to allow the liquid to flow through the suction conduit and the illumination chamber.

Also in the case, the same effects as the effects of the above-described first embodiment can be obtained.

In addition, in the fourth embodiment, each of the flow paths 9Dx communicates with the suction conduit having a diameter larger than the diameter of the liquid feeding conduit.

With the configuration, the flow rate of the liquid flowing through the conduit can be increased, so that the liquid can be introduced into the illumination chamber more efficiently.

Furthermore, in the fourth embodiment, the presence of impurities and the like contained in the object to be suctioned such as a liquid in the body cavity is taken into consideration.

Therefore, the filter 35 is provided between the flow path 9Dx and the suction conduit (suction path through hole 9e), which is the second conduit.

In addition to the configuration, the interval T1 of the gap 9c in the illumination chamber is set to 0.1 mm or less.

With the configurations, it is possible to prevent impurities and the like contained in the object to be suctioned, such as a liquid in the body cavity, from flowing into the illumination chamber (non-through hole 9a), into the gap 9b, during suction.

Note that the above illustration has the configuration such that each of the filter 35 is provided in the flow path 9Dx and the interval T1 of the gap 9c in the illumination chamber is set to 0.1 mm or less.

However, the present disclosure is not limited to the configuration, and may be configured by using either of the filter 35 and the setting of the interval T1 of the gap 9c.

The first to fourth embodiments described above each illustrate a configuration of the distal end portion each provided with flow paths in which either of the liquid feeding conduit or the suction conduit communicates with the illumination chambers to allow the liquid to flow through the liquid feeding conduit or the suction conduit and the illumination chambers.

In a fifth embodiment described below illustrates a configuration of a distal end portion in which flow paths provided between the outside of an insertion portion and illumination chambers to communicate with each other to allow the liquid to flow through the insertion portion and the illumination chambers.

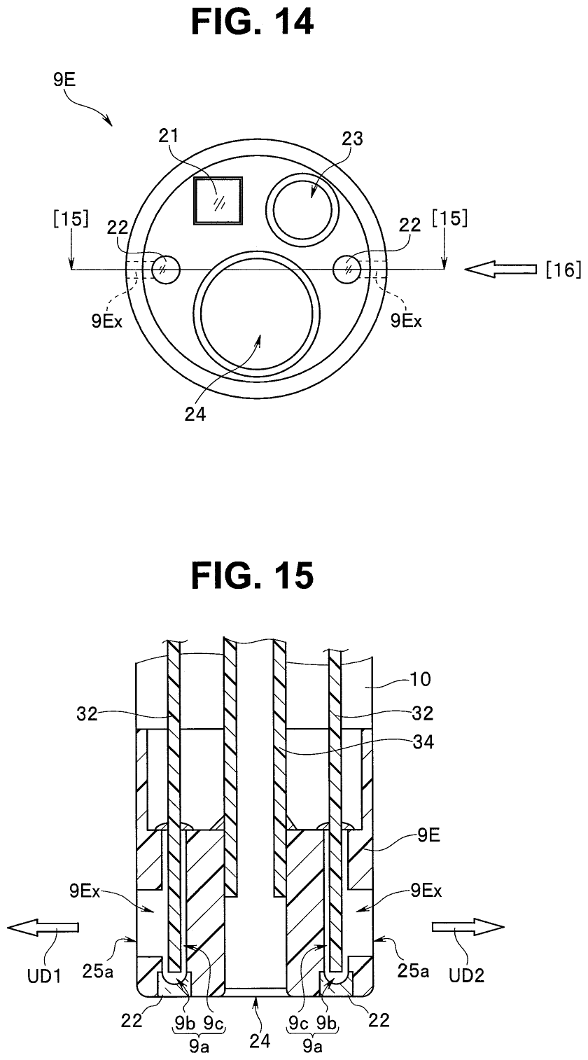

An endoscope of the fifth embodiment of the present disclosure is to be explained below with FIGS. 14 to 16.

FIG. 14 is a plan view of the distal end surface of the distal end portion of the insertion portion in the endoscope, which is an insertion apparatus of the fifth embodiment of the present disclosure.

FIG. 15 is a cross-sectional view taken along a line [15]-[15] in FIG. 14.

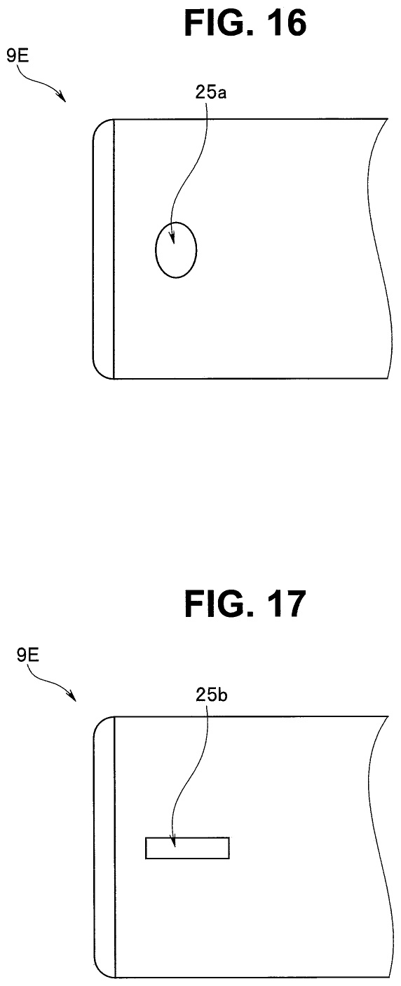

FIG. 16 is a plan view of a side surface of the distal end portion as seen in the direction of an arrow mark [16] in FIG. 14.

FIG. 17 is a diagram showing a modification of a flow path openings of the distal end portion of the fifth embodiment of the present disclosure.

FIG. 17 is a side view corresponding to FIG. 16 of the modification.

The basic components of the distal end portion of the insertion portion (distal end portion 9E) in the endoscope of the fifth embodiment of the present disclosure are substantially the same as the basic components of each of the above-described embodiments.

The fifth embodiment differs in the form of the flow paths formed inside the distal end portion.

Therefore, the same reference numerals and characters are to be given and the descriptions are to be omitted for the same components as the components of each of the above-described embodiments, and the descriptions are to be made below for the different components.

In the endoscope of each of the above-described embodiments, the flow paths are provided in which either of the liquid feeding conduit or the suction conduit communicates with the inside of the illumination chambers to allow the liquid to flow through the liquid feeding conduit or the suction conduit and the inside of the illumination chambers.

Instead of the configuration, the endoscope of the fifth embodiment differs in that the distal end portion 9E has second flow paths 9Ex configured to allow the outer surface of the distal end portion 9E to communicate with the inside of the illumination chambers to allow the liquid to flow through the outer surface of the distal end portion 9E and the inside of the illumination chambers.

As shown in FIG. 14 and the like, the second flow paths 9Ex allow the lateral side outer surfaces of the distal end portion 9E to communicate with the inside of the illumination chambers (non-through hole 9a). The illumination chamber is configured to fluidly communicate with the peripheral part of the insertion portion 6 via the second opening 9Ex.

For the purpose, as shown in FIG. 16, sites of the side surface of the distal end portion 9E has flow path openings 25a.

For example, as shown in FIG. 16, each of the flow path openings 25a is formed in a circular or elliptical shape.

In addition, the form of the flow path opening is not limited to the illustration shown in FIG. 16.

For example, as shown in FIG. 17, the flow path opening may be a rectangular flow path opening 25b.

The fifth embodiment is configured so that the liquid in the body cavity is introduced into the illumination chambers.

Therefore, it may be considered that impurities and the like contained in the liquid in the body cavity enter the illumination chambers (the gap 9b).

As an example, it can similarly apply the configuration applied to the above-described fourth embodiment.

In other words, each of the gaps 9c in the illumination chambers is configured to have an interval (T1; not shown in FIG. 15; see FIG. 12) set to 0.1 mm or less.

In addition, each of the second flow paths 9Ex is configured to have a filter 35 provided at a position between the flow path opening 25a (25b) and the illumination chamber.

It can apply at least either one of the two configurations.

Note that the second flow paths 9Ex and flow path openings 25a (25b) are respectively provided for the two light emitting portions 32x.

Further, for example, when the insertion portion of the endoscope is operated to bend, a plane orthogonal to the moving direction of the distal end portion is set.

In the case, the surface of each flow path opening 25a (25b) can be disposed parallel to the plane.

With such a configuration, operation of the endoscope to bend can actively introduce the liquid into the second flow path 9Ex from the flow path opening 25a (25b).

In other words, arrow marks UD1 and UD2 in FIG. 15 conceptually indicate the moving direction of the distal end portion when the bending operation is performed.

As shown in FIG. 15, the plane on which each flow path opening 25a (25b) can be disposed be set parallel to the plane orthogonal to the line connecting the arrow marks UD1 and UD2.

As described above, according to the fifth embodiment, it is possible to obtain the same effects as effects of each of the above embodiments.

Furthermore, according to the fifth embodiment, the liquid can be forcibly introduced into the illumination chambers only by performing the bending operation that is performed during normal use, without performing the liquid feeding operation or the suction operation.

Therefore, it is possible to obtain stable and illumination light distribution characteristics with a simple configuration of only providing the second flow paths 9Ex between the outer surfaces and the illumination chambers. The insertion portion 6 includes the peripheral surface, the illumination chamber, the opening 9Ex, the optical fiber 32 configured to guide and radiate the illumination light, and the illumination window 22 configured to radiate the illumination light to the outside of the insertion portion 6. The opening 9Ex is located in the peripheral surface and the illumination chamber is in fluid communication with the outside of the insertion portion 6 via the opening. The distal end of the optical fiber 32 is located inside the illumination chamber.

Next, an endoscope of a sixth embodiment of the present disclosure is to be explained below.

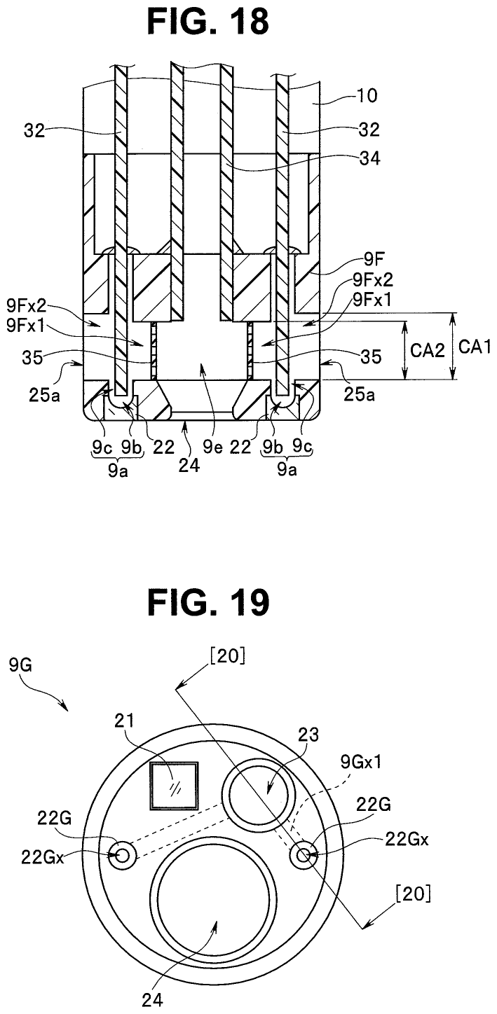

FIG. 18 is a cross-sectional view showing an enlarged distal end portion of an insertion portion in the endoscope, which is an insertion apparatus according to the sixth embodiment of the present disclosure.

The basic components of the distal end portion of the insertion portion (distal end portion 9F) in the endoscope of the sixth embodiment of the present disclosure are substantially the same as the basic components of the fifth embodiment described above.

The sixth embodiment differs in the form of the flow paths formed inside the distal end portion 9F.

Therefore, the same reference numerals and characters are to be given and the descriptions are to be omitted for the same components as the components of each of the above-described embodiments, and the descriptions are to be made below for the different components.

In the endoscope of the sixth embodiment, the distal end portion 9F has first flow paths 9Fx1 each having the same form as the flow path 9Dx (see FIG. 13) in the fourth embodiment described above.

Further, in the endoscope of the sixth embodiment, the distal end portion 9F has second flow paths 9Fx2 each having the same form as the second flow path 9Ex (see FIG. 15) in the fifth embodiment described above.

Here, each of the first flow paths 9Fx1 is a flow path that allows the suction conduit to communicate with the inside of each illumination chamber to allow the liquid or the like to flow through the suction conduit and the inside of each illumination chamber.

In addition, each of the second flow paths 9Fx2 is a flow path that allows the outer surface of the distal end portion 9F to communicate with the inside of the illumination chamber to allow the liquid or the like to flow through the outer surface of the distal end portion 9F and the inside of the illumination chamber.

Each of the first flow paths 9Fx1 communicates with the second flow path 9Fx2.

In the case, the first flow path 9Fx1 and the second flow path 9Fx2 have different cross-sectional areas.

Here, for example, if the cross-sectional area of the second flow path 9Fx2 is smaller than the cross-sectional area of the first flow path 9Fx1, fluid pressure is applied to the first flow path 9Fx1 by the liquid or the like that flows into the first flow path 9Fx1 from the suction conduit during suction.

Here, for example, the cross-sectional area of the first flow path 9Fx1 means the cross-sectional area of the area indicated by a reference character CA1 in FIG. 18.

The cross-sectional area of the second flow path 9Fx2 means the cross-sectional area of the area indicated by a reference character CA2 in FIG. 18.

Therefore, there is an advantage in which the liquid can be easily introduced into the gap 9b. The insertion portion 6 includes a second conduit having a third opening and a fourth opening. The third opening is on a peripheral part of the insertion portion 6, and the fourth opening is on a peripheral part of the illumination chamber. The second conduit is in fluid communication with the illumination chamber and the outside of the insertion portion 6 via the third opening and the second opening.

Contrarily, it is assumed that the cross-sectional area (CA2) of the second flow path 9Fx2 is larger than the cross-sectional area (CA1) of the first flow path 9Fx1 (CA1<CA2).

In such a case, the liquid or the like that flows into the first channel 9Fx1 from the suction conduit during suction tends to flow out from the first flow path 9Fx1 to the second flow path 9Fx2.

In the case, there is an advantage in which air bubbles or the like generated in the liquid or the like by suction do not stagnate in the illumination chamber and are easily discharged as the liquid flows.

Note that the first flow path 9Fx1 and the second flow path 9Fx2 each can have an interval of the gap 9c formed with the optical fiber cable 32 which interval is ensured to be 0.1 mm or more.

In such a way, the liquid or the like can be introduced into the illumination chamber.

A filter 35 is provided between the first flow path 9Fx1 and the suction conduit (suction path through hole 9e), as in the above-described fourth embodiment.

With such a configuration, in the sixth embodiment, part of the liquid or the like that flows from the channel opening 24 into the suction conduit flows into the first flow path 9Fx1 during suction.

Then, the gap 9b is filled with the liquid or the like.

Furthermore, among part of the liquid or the like introduced into the suction conduit through the channel opening 24 during suction, some liquid or the like flows from the first flow path 9Fx1 to the second flow path 9Fx2 through (the gap 9c in) the illumination chamber.

Then, among the part of the liquid or the like described above, some liquid or the like flows out from the flow path opening 25a to the outside of the distal end portion 9F.

In contrast, in a bending operation or the like, some part of the liquid or the like introduced from the flow path opening 25a into the second channel 9Fx2 contributes to filling the gap 9b.

At the same time, the other part of liquid or the like flows from the second flow path 9Fx2 to the first flow path 9Fx1 through (the gap 9c in) the illumination chamber, and then flows through the suction conduit.

As described above, according to the sixth embodiment, it is possible to obtain the same effects as the effects of each of the above embodiments.

Furthermore, according to the sixth embodiment, the first flow path 9Fx1 and the second flow path 9Fx2 are provided, so that liquid or the like can flow between the inside and the outside of the distal end portion 9F.

Such a configuration allows the liquid or the like to be efficiently introduced into the gap 9b without allowing air bubbles or the like to stagnate in the gap 9b.

Therefore, it is possible to obtain stable and illumination light distribution characteristics.

Furthermore, the first flow path 9Fx1 and the second flow path 9Fx2 allow liquid and the like to flow between the inside and the outside of the distal end portion 9F.

Therefore, the size of each flow path itself can be reduced.

Therefore, it is possible to contribute to downsizing of the distal end portion and further downsizing of the endoscope itself.

Note that, in the sixth embodiment, the first flow path 9Fx1 is configured to allow the suction conduit to communicate with the inside of the illumination chamber to allow the liquid to flow through the suction conduit and the inside of the illumination chamber, but any other configuration may be used.

For example, as in the configuration example shown in the first embodiment, the first flow path may be configured to allow the liquid feeding conduit to communicate with the inside of the illumination chamber to allow the liquid to flow through the liquid feeding conduit and the inside of the illumination chamber.

If such a configuration is used, the same effects as the effects of the sixth embodiment can also be obtained.

Next, an endoscope according to a seventh embodiment of the present disclosure is to be described below with FIGS. 19 to 21.

FIG. 19 is an enlarged plan view showing a distal end surface of a distal end portion of an insertion portion in the endoscope, which is an insertion apparatus of the seventh embodiment of the present disclosure.

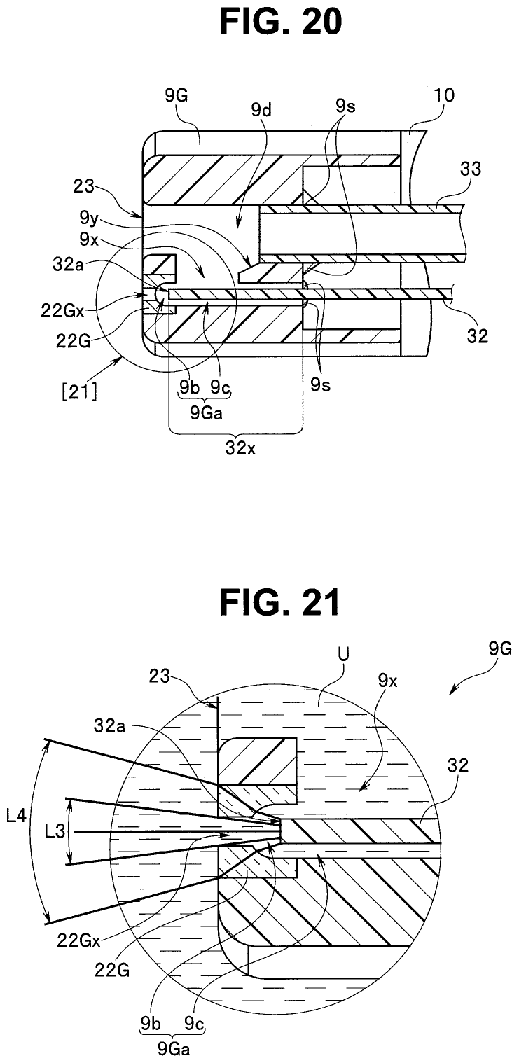

FIG. 20 is a cross-sectional view taken along a line [20]-[20] in FIG. 19.

FIG. 21 is an enlarged cross-sectional view of a main part showing an enlarged cut-out area indicated by an arrow mark [21] in FIG. 20.

FIG. 21 conceptually shows rays of illumination light that illuminate the inside of the body cavity of the subject in the endoscope of the seventh embodiment of the present disclosure.

The basic components of the distal end portion of the insertion portion (distal end portion 9G) in the endoscope of the seventh embodiment of the present disclosure are substantially the same as the basic components of the first embodiment described above.

The seventh embodiment slightly differs in the form of illumination windows 22G provided on the distal end surface of the distal end portion 9G.

Therefore, the same reference numerals and characters are to be given and the descriptions are to be omitted for the same components as the components of the above-described first embodiment, and the descriptions are to be made below for the different components.

In the endoscope of the seventh embodiment, the distal end portion 9G is provided with an observation window 21, the illumination windows 22G, a liquid feeding opening 23, a channel opening 24, and the like on the distal end surface.

Among the windows and openings, the observation window 21, the illumination windows 22G, and the liquid feeding opening 23 are configured in the same manner as the ones in the above-described first embodiment.

In contrast, each of the illumination windows 22G differs in that an illumination window through hole 22Gx is formed in a substantially central area.

The illumination window through hole 22Gx allows the outside of the distal end surface side of the distal end portion 9G to communicate with the inside of the illumination chamber 9Ga.

As a result, the illumination window through hole 22Gx functions as a third flow path that allows a fluid to flow between the outside of the distal end portion 9G and the inside of the illumination chamber 9Ga.

In other words, the illumination window 22G configuring part of the illumination lens is provided with the illumination window through hole 22Gx.

The illumination window through hole 22Gx is the third flow path that allows the liquid in the illumination chamber 9Ga to pass to the outside.

Further, the illumination window through hole 22Gx is provided at a position facing the distal end surface 32a of the optical fiber cable 32 in the light emitting portion 32x.

Here, the illumination chamber 9Ga is a space that includes a gap 9b that functions as part of the illumination lens and accommodates the light emitting portion 32x.

In the first embodiment and the like described above, the illumination chamber is defined as the internal space of the non-through hole 9a.

Instead of such configuration, in the seventh embodiment, the illumination chamber 9Ga is provided with an illumination window through hole 22Gx to be formed to pass through the distal end portion 9G.

In the respect, the configuration of the seventh embodiment differs from the configurations of the above-described first embodiment and the like.

Other configurations are substantially the same as the configurations of the above-described first embodiment.

In the seventh embodiment configured as described above, the illumination window through hole 22Gx, which is the third flow path, is provided in the substantially central area of the illumination window 22G.

The configuration allows the inside of the illumination chamber to communicate with the outside of the distal end portion 9G.

The inside of the illumination chamber communicates with the flow path 9x.

Therefore, with the configuration, the liquid flowing from the liquid feeding conduit toward the distal end side flows from the liquid feeding path through hole 9d through the flow path 9x into the illumination chamber 9Ga.

Part of the liquid that has thus flowed into the illumination chamber 9Ga fills the gap 9b, while some of the liquid passes through the illumination window through hole 22Gx and flows out to the outside on the distal end side of the distal end portion 9G.

At the time, if the liquid continuously flows in from the liquid feeding conduit, the gap 9b is kept filled with the liquid.

In addition, the liquid flowing through the illumination window through hole 22Gx from the flow path 9x through the illumination chamber 9Ga flows.

Therefore, the liquid that fills the gap 9b can be clean liquid.

Also, at the time, among the illumination light radiated forward from the distal end surface 32a of the optical fiber cable 32, the illumination light emitted from the vicinity of the substantially central area (hereinafter referred to as central illumination light; see a reference character L3 in FIG. 21) passes through the illumination window through hole 22Gx and is radiated forward.

At the time, the central illumination light L3 passes through the liquid filled in the gap 9b and the illumination window through hole 22Gx.

In the case, the liquid filling the gap 9b and the illumination window through hole 22Gx is transparent and has a higher refractive index than air, so that the central illumination light L3 is refracted in the diffusion direction.

Therefore, the central illumination light L3 passes through the liquid to contribute to light distribution in the peripheral direction.

In contrast, among the illumination light radiated forward from the distal end surface 32a of the optical fiber cable 32, the illumination light emitted from the peripheral area (hereinafter referred to as peripheral illumination light; see a reference character L4 in FIG. 21) passes through the peripheral area of the inner surface (curved surface with a radius of curvature) of the illumination window 22G and is radiated forward.

At the time, the peripheral illumination light L4 is refracted in the diffusion direction by the inner surface of the illumination window 22G.

Thereby, a more extensive illumination light distribution can be obtained.

In view of such actions, it can set the illumination window through hole 22Gx to about half the diameter of the illumination window 22 in order to obtain an extensive illumination light distribution.

As described above, according to the seventh embodiment, it is possible to obtain the same effects as the effects of each of the above embodiments.

Furthermore, according to the seventh embodiment, the illumination window through hole 22Gx is provided in the illumination window 22G, and thereby the liquid passes between the flow path 9x and the outside.

With the configuration, it is possible to efficiently introduce the liquid or the like into the gap 9b while preventing the stagnation of air bubbles or the like in the gap 9b.

Therefore, it is possible to obtain stable and illumination light distribution characteristics. The illumination lens includes the illumination window 22 extending from the distal end surface of the insertion portion 6 to the distal end surface of the illumination chamber. The insertion portion 6 further includes a second conduit 22Gx having a third opening. The second conduit 22Gx extends through the illumination window 22, and the second conduit 22Gx is in fluid communication with the illumination chamber and the outside of the insertion portion 6 via the third opening.

The endoscope, which is the insertion apparatus shown in each of the above embodiments, can be applied to, for example, a single-use endoscope to be disposed of after being used once, but may be a re-use endoscope to be repeatedly used.

In the case, the configuration itself is the same as in each of the above-described embodiments.

In the configuration of each of the above-described embodiments, the distal end portion tends to have a complicated internal configuration due to the configuration in which the flow paths that communicate with the conduit is provided.

However, in such a case of a single-use endoscope, even if the distal end portion or the like has a complicated configuration, there is no need to perform operations such as strict sterilization and cleaning after use.

Furthermore, the single-use endoscope can be disposed of after use, resulting in efficient operation.

Note that the endoscope of each of the above-described embodiments may also be a reusable conventional reusable endoscope.

As described above, in each of the above-described embodiments, there are provided flow paths each configured to allow the conduit inserted through the inside of the distal end portion to communicate with the inside of the illumination chambers to allow the liquid to flow through the conduit and the inside of the illumination chambers.

As a result, the embodiment has a configuration such that the liquid is forced to flow into the illumination chambers.

The configuration achieves a configuration in which part of the area (gap 9b) in each of the illumination chamber functions as part of the illumination lens.

However, the present disclosure is not limited to the configuration examples illustrated in each of the above-described embodiments.

For example, each of the illumination chamber, which is a space including the gap 9b functioning as part of the illumination lens and accommodating the light emitting portion 32x, is filled with liquid in advance.

In such a state, the illumination chamber may be configured as a sealed space.

In other words, the endoscope, which is an insertion apparatus, is configured so that the inside of the illumination chamber has liquid filled between the illumination lens and the light emitting portion.

The endoscope includes: an insertion portion inserted into a subject; an illumination portion that includes the illumination lens and the light emitting portion and illuminates the inside of the subject; a distal end portion of the insertion portion having the illumination chamber including the illumination lens and accommodating the light emitting portion.

Also with such a configuration, substantially the same effects as the effects of each of the above-described embodiments can be obtained.

A method of illuminating with the insertion apparatus comprises flowing a liquid through the first conduit and through the second opening into the illumination chamber, filling the illumination chamber to a level with the liquid, and radiating the illumination light to the outside of the insertion portion 6. The level is sufficient to optically couple the distal end of the optical fiber 32 to an illumination window 22 of the illumination lens. The radiating may include radiating the illumination light to the outside of the insertion portion 6 illuminates an inside of the subject.

A medical kit comprises: the insertion apparatus including the insertion portion 6 configured for insertion into the subject. The insertion portion 6 includes: the illumination chamber, the optical fiber 32 configured to guide and radiate the illumination light, the illumination window 22 configured to radiate the illumination light to the outside of the insertion portion 6. The distal end of the optical fiber 32 is located inside of the illumination chamber. The illumination window 22 is spaced apart from the distal end of the optical fiber 32 to form the space. The medical kit can comprise a liquid located in the space. The liquid may have such as viscosity, optical properties, refractive index, etc. The liquid (such as physiological saline) may have a refractive index of 1.33 and the material of the illumination window 22 (part of the illumination lens) may have a refractive index of 1.63.

It goes without saying that the present disclosure is not limited to the above-described embodiments and various modifications and applications can be implemented without departing from the gist of the disclosure.

Furthermore, the above-described embodiments include disclosures at various stages, and various disclosures can be extracted by appropriately combining a plurality of disclosed constituent elements.

For example, even if some constituent elements are deleted from all the constituent elements shown in the above one embodiment, a configuration from which the constituent elements are deleted can be extracted as a disclosure as long as the problem to be solved by the disclosure can be solved and the effects of the disclosure can be obtained.

Furthermore, components across different embodiments may be combined as appropriate.

The disclosure is not restricted by the particular aspects except as limited by the appended claims.

Example 1. An insertion apparatus comprising:
an insertion portion inserted into a subject;
at least one conduit that is inserted through the insertion portion and is configured to allow a liquid to flow between one end side and another end side;
an illumination portion that includes an illumination lens and a light emitting portion and is configured to illuminate an inside of the subject; and
a distal end portion of the insertion portion provided on a distal end side of the insertion portion, the distal end portion including an illumination chamber and a flow path, the illumination chamber including the illumination lens, the illumination chamber accommodating the light emitting portion, the flow path being configured to allow the liquid to flow through one end side of the conduit, an inside of the conduit, and an inside of the illumination chamber.
   Example 2. The insertion apparatus according to Example 1, wherein
the flow path allows the liquid to flow between the illumination lens and the light emitting portion.
   Example 3. The insertion apparatus according to Example 1, wherein
a distal end portion cover is further provided on a distal end side of the distal end portion of the insertion portion, at least part of the distal end portion cover being transparent, and
the illumination lens is integrally formed with the distal end portion cover.
   Example 4. The insertion apparatus according to Example 1, wherein
the at least one conduit includes at least:
a first conduit configured to allow the liquid to flow from an outside of the insertion portion toward an inside of the subject; and
a second conduit, different from the first conduit, configured to allow the liquid to flow from an inside of the subject toward an outside of the insertion portion.
   Example 5. The insertion apparatus according to Example 4, wherein
the second conduit has a larger inner diameter than the first conduit, and
the flow path is connected only to the second conduit in the distal end portion of the insertion portion.
   Example 6. The insertion apparatus according to Example 5, wherein
a filter is provided between the flow path and the second conduit.
   Example 7. The insertion apparatus according to Example 5, wherein
the second conduit also serves as a treatment instrument channel configured to allow insertion of a treatment instrument from an outside of the insertion portion toward an inside of the subject.
   Example 8. The insertion apparatus according to Example 4, wherein
the flow path is connected only to the first conduit in the distal end portion of the insertion portion and a boundary between the flow path and the first conduit is provided with a slope formed in a direction to expand the flow path toward a distal end.
   Example 9. The insertion apparatus according to Example 4, wherein
a pump configured to forcibly cause the liquid to flow through is connected to at least either of the other end side of the first conduit or the other end side of the second conduit.
   Example 10. The insertion apparatus according to Example 1, wherein
the distal end portion of the insertion portion includes: a first distal end portion provided on the distal end side; and a second distal end portion connected to the first distal end portion and provided on a proximal end side, the second distal end portion being configured as a separate component from the first distal end portion, and
the illumination lens is provided on a side of the first distal end portion.
   Example 11. The insertion apparatus according to Example 10, wherein
the flow path is provided on the side of the first distal end portion.
   Example 12. The insertion apparatus according to Example 10, wherein
the second distal end portion has a watertight structure, on the proximal end side of the light emitting portion, to make a connection with an inner surface of the illumination chamber watertight.
   Example 13. The insertion apparatus according to Example 1, wherein
the distal end portion of the insertion portion has a watertight structure, on the proximal end side of the light emitting portion, to make a connection with an inner surface of the illumination chamber watertight.
   Example 14. The insertion apparatus according to Example 1, wherein
the light emitting portion includes a distal end side of an optical fiber cable configured to guide illumination light from an outside, and
a gap provided between an inner surface of the illumination chamber and a side surface of the optical fiber cable is 0.1 mm or less.
   Example 15. The insertion apparatus according to Example 1, wherein
the distal end portion of the insertion portion further includes a second flow path configured to allow an outer surface of the distal end portion of the insertion portion to communicate with an inside of the illumination chamber.
   Example 16. The insertion apparatus according to Example 15, wherein
the flow path and the second flow path have different cross-sectional areas.
   Example 17. The insertion apparatus according to Example 1, wherein
in the illumination portion, a shape of the illuminating lens is set to exhibit highest light distribution characteristics in a case in which a medium between the light emitting portion and the illumination lens has a refractive index of 1.33 and a material of the illumination lens has a refractive index of 1.63.
   Example 18. The insertion apparatus according to Example 1, wherein
the flow path is provided with a structure such that, when a flow of the liquid from the conduit flows through the flow path, the liquid flows more easily on an upstream side of the conduit than on a downstream side of the conduit.
   Example 19. The insertion apparatus according to Example 1, wherein
the illumination lens is further provided with a third flow path at a position facing a distal end surface of the light emitting portion, the third flow path allowing the liquid in the illumination chamber to pass to an outside.
   Example 20. The insertion apparatus according to Example 1, wherein
the insertion apparatus is an endoscope or a single-use endoscope, the single-use endoscope being disposed of after only a single use.
   Example 21. An insertion apparatus comprising:
an insertion portion inserted into a subject;
an illumination portion that includes an illumination lens and a light emitting portion and is configured to illuminate an inside of the subject;
a distal end portion of the insertion portion, the distal end portion including an illumination chamber, the illumination chamber including the illumination lens and accommodating the light emitting portion; and
a liquid filled between the illumination lens and the light emitting portion inside the illumination chamber.
   Example 22. A method of illuminating an inside of a subject, the method comprising:
allowing a liquid to flow between one end side and another end side of a conduit inserted through an insertion portion inserted into the subject;
allowing the liquid flowing through the conduit to flow into a flow path in a distal end portion of the insertion portion, the distal end portion being provided on a distal end side of the insertion portion, the distal end portion including: an illumination chamber; the one end side of the conduit; and the flow path, the illumination chamber accommodating an illumination portion including an illumination lens and a light emitting portion, the flow path communicating with an inside of the illumination chamber;
filling the liquid in the illumination chamber; and
emitting illumination light from the illumination portion to illuminate an inside of the subject.

What is claimed is:

1. An insertion apparatus, comprising:
an insertion portion including:
a first conduit having a first opening, wherein the first conduit is in fluid communication with an outside of the insertion portion via the first opening,
an illumination chamber having a second opening, wherein the illumination chamber is configured to fluidly communicate with a peripheral part of the first conduit via the second opening,
an optical fiber configured to guide and radiate an illumination light, wherein a distal end of the optical fiber is located inside of the illumination chamber, and
an illumination lens provided distally relative to the distal end of the optical fiber, wherein the illumination lens is configured to radiate the illumination light to the outside of the insertion portion.

2. The insertion apparatus according to claim 1, wherein the illumination lens includes an illumination window,
wherein the illumination window is spaced apart from the distal end of the optical fiber to form a space, and
wherein the illumination chamber is configured to store a liquid in the space.

3. The insertion apparatus according to claim 2, wherein the liquid has a refractive index of 1.33,
wherein the illumination lens includes an illumination window extending from a distal end surface of the insertion portion to a distal end surface of the illumination chamber, and
wherein the illumination window has a refractive index of 1.63.

4. The insertion apparatus according to claim 1, wherein the first conduit is configured to suction a fluid through the first opening and toward a proximal end of the first conduit.

5. The insertion apparatus according to claim 4, wherein the insertion portion further includes:
a discharge conduit having a discharge opening, wherein the discharge conduit is in fluid communication with the outside of the insertion portion via the discharge opening, and
wherein the first conduit has a first inner diameter, the discharge conduit has a second inner diameter, and the second inner diameter is smaller than the first inner diameter.

6. The insertion apparatus according to claim 4, wherein a filter is located in the second opening.

7. The insertion apparatus according to claim 5, wherein the first conduit is configured for insertion of a treatment instrument.

8. The insertion apparatus according to claim 1, wherein the first conduit includes a guide part configured to guide a liquid from the first conduit toward the illumination chamber.

9. The insertion apparatus according to claim 8, wherein the guide part has an inclined surface provided around at least a portion of a periphery of the second opening.

10. The insertion apparatus according to claim 8, wherein the guide part has a recessed surface provided distally relative to the second opening, wherein the recessed surface is recessed radially.

11. The insertion apparatus according to claim 1, wherein the illumination chamber is connected to an optical fiber conduit,
wherein portions of the optical fiber are located in the optical fiber conduit, and
wherein a gap between an inner surface of the optical fiber conduit and the optical fiber is sealed to form a watertight structure.

12. The insertion apparatus according to claim 1, wherein the illumination chamber includes an inner surface,
wherein the optical fiber includes an outer surface, and
wherein a maximum distance between the outer surface of the optical fiber and the inner surface of the illumination chamber is 0.1 mm.

13. The insertion apparatus according to claim 1, wherein the insertion portion further includes a second conduit having a third opening,
wherein the third opening is on a peripheral part of the insertion portion,
wherein the second conduit is in fluid communication with the illumination chamber, and
wherein the second conduit is in fluid communication with the outside of the insertion portion via the third opening.

14. The insertion apparatus according to claim 1, wherein the illumination lens includes an illumination window extending from a distal end surface of the insertion portion to a distal end surface of the illumination chamber,
wherein the insertion portion further includes a second conduit having a third opening,
wherein the second conduit extends through the illumination window, and
wherein the second conduit is in fluid communication with the illumination chamber and the outside of the insertion portion via the third opening.

15. The insertion apparatus according to claim 1, wherein the insertion apparatus is an endoscope or a single-use endoscope.

16. A method of illuminating with the insertion apparatus according to claim 1, the method comprising:
flowing a liquid through the first conduit and through the second opening into the illumination chamber;
filling the illumination chamber to a level with the liquid; and
radiating the illumination light to the outside of the insertion portion,
wherein the level is sufficient to optically couple the distal end of the optical fiber to an illumination window of the illumination lens.

17. The method according to claim 16, wherein radiating the illumination light to the outside of the insertion portion illuminates an inside of a subject.

18. An insertion apparatus, comprising:
an insertion portion configured for insertion into a subject,
wherein the insertion portion includes:
a peripheral surface,
an illumination chamber and an opening, wherein the opening is located in the peripheral surface and wherein the illumination chamber is in fluid communication with an outside of the insertion portion via the opening,
an optical fiber configured to guide and radiate an illumination light, wherein a distal end of the optical fiber is located inside the illumination chamber, and
an illumination window configured to radiate the illumination light to the outside of the insertion portion.

19. A medical kit, comprising:
an insertion apparatus including an insertion portion configured for insertion into a subject,
wherein the insertion portion includes:
an illumination chamber,
an optical fiber configured to guide and radiate an illumination light, wherein a distal end of the optical fiber is located inside of the illumination chamber, and
an illumination window configured to radiate the illumination light to an outside of the insertion portion, and
wherein the illumination window is spaced apart from the distal end of the optical fiber to form a space.

20. The medical kit according to claim 19, further comprising a liquid,
wherein the liquid is located in the space.
